(19) United States
(12) Patent Application Publication
(10) Pub. No.: US 2023/0314438 A1
(43) Pub. Date: Oct. 5, 2023

(54) SECRETED PHOSPHORYLATED HEAT SHOCK PROTEIN-70 AS A BIOMARKER FOR TREATING AND DIAGNOSING CANCER

(71) Applicant: University of Cincinnati, Cincinnati, OH (US)

(72) Inventors: Xiaoyang Qi, Cincinnati, OH (US); Subrahmanya Duttu Vallabhapurapu, Cincinnati, OH (US)

(73) Assignee: University of Cincinnati, Cincinnati, OH (US)

(21) Appl. No.: 17/915,789

(22) PCT Filed: Apr. 2, 2021
(86) PCT No.: PCT/US2021/025483

§ 371 Date: Sep. 29, 2022

Related U.S. Application Data
(60) Provisional application No. 63/004,677, filed on Apr. 3, 2020.

(51) Int. Cl. G01N 33/574

(52) U.S. Cl. CPC G01N 33/57488; G01N 2333/46; G01N 2440/14; G01N 2800/52

(57) ABSTRACT

A method of non-invasively diagnosing and treating a cancer characterized by high surface phosphatidylserine (PS) expression in a subject in need thereof is provided, the method including: obtaining a liquid biological sample from the subject; detecting a presence of cancer-secreted soluble phosphorylated Heat shock protein-70 (Hsp70) in the liquid biological sample; diagnosing the subject with a cancer characterized by high surface PS expression when cancer-secreted soluble phosphorylated Hsp70 is present in the liquid biological sample; and administering an anti-cancer therapy targeted to high surface PS expressing-cancers to the diagnosed subject. Also provided is a method for monitoring therapeutic efficacy of a treatment in a subject with a cancer characterized by high surface PS expression.

### CROSS REFERENCE TO RELATED APPLICATION

This application claims priority to U.S. Provisional Application No. 63/004,677 filed Apr. 3, 2020, the contents of which are incorporated herein by reference in their entirety.

### TECHNICAL FIELD

This disclosure relates to diagnosis and treatment of cancer. Specifically, the present disclosure relates to methods of non-invasive diagnosis and treatment of cancers expressing high levels of surface phosphatidylserine via detection of cancer-secreted Heat shock protein-70 (Hsp70).

### BACKGROUND

Phosphatidylserine (PS) is localized in the inner leaflet of the lipid bilayer of normal eukaryotic cells. PS exposure on the external surface of an apoptotic cell provides a signal marking the cell for phagocytosis. In non-apoptotic cells, surface PS contributes to coagulation, myoblast fusion, and immune regulation. In viable cancer cells, surface exposure of PS is elevated compared to normal cells and contributes to the immunosuppressive tumor microenvironment (TME), which in turn promotes cancer growth. Accordingly, development of PS-targeted immune therapies is considered a promising area of anti-cancer drug discovery. Several anti-PS therapies are currently in clinical and pre-clinical development, including the monoclonal antibody bavituximab (also known as PGN401) and Saposin C-dioleoylphosphatidylserine (SapC-DOPS).

Among cancers and cancer patients, the level of surface PS on cancer cells varies. The higher the level of surface PS in a cancer cell, the more likely the cancer cell is to respond to anti-cancer therapeutics targeted to PS. At present, it is challenging to determine whether a particular cancer is characterized by high surface PS, since any direct determination would require an invasive biopsy and analysis of the cancer cells of interest. Biopsy and analysis is not always feasible and, even when possible, may be traumatic or painful for the cancer patient.

A need exists for a non-invasive method of identifying cancers that express high levels of surface PS and would thus be candidates for treatment with PS-targeted anti-cancer therapies.

### SUMMARY

The present inventors have found that cancer-secreted soluble, non-membrane bound Heat shock protein-70 (Hsp70), and in particular phosphotyrosine Hsp70 (py-Hsp70), correlates positively with surface PS localization in the tumor microenvironment and serves as a useful biomarker for identifying cancers susceptible to PS-targeted therapy.

Accordingly, in one embodiment, a method of non-invasively diagnosing and treating a cancer characterized by high surface phosphatidylserine (PS) expression in a subject in need thereof is provided, the method comprising: obtaining a liquid biological sample from the subject; detecting a presence of cancer-secreted soluble phosphorylated Heat shock protein-70 (Hsp70) in the liquid biological sample; diagnosing the subject with cancer characterized by high surface PS expression when cancer-secreted soluble phosphorylated Hsp70 is present in the liquid biological sample; and administering an anti-cancer therapy targeted to high surface PS expressing-cancers to the diagnosed subject.

In another embodiment, a method is provided for monitoring the therapeutic efficacy of a treatment in a subject with a cancer characterized by high surface phosphatidylserine (PS) expression, the method comprising: treating the subject with an anti-cancer therapy targeted to high surface PS expressing-cancers; measuring the level of cancer-secreted soluble tyrosine-phosphorylated Hsp70 (py-Hsp70) in a liquid biological sample obtained from the subject after administering the anti-cancer therapy; and altering the dosage of the anti-cancer therapy, the frequency of dosing the anti-cancer therapy, or the course of therapy administered to the subject based on the level of cancer-secreted soluble py-Hsp70 measured.

These and other objects, features, embodiments, and advantages will become apparent to those of ordinary skill in the art from a reading of the following detailed description and the appended claims.

### BRIEF DESCRIPTION OF THE FIGURES

FIG. 1 shows that induction of macrophage M2 polarization by cancer CM is neutralizable by monoclonal antibodies to Hsp70. (A) Co-culture of THP-1 cells together with the CFSE labeled Gli36 cells was followed by measurement of the differentiation marker CD14 on CFSE negative THP-1 cells by flow cytometry. (B) Conditioned medium from indicated cell lines was incubated with THP-1 cells and CD14 expression was measured by flow cytometry. (C) Flow cytometric measurement of macrophage M2 markers, CD206 and CD301 in J774 macrophage cells in response to DMEM, AsPC-1 CM or Gli36 CM. (D) Macrophage M2 polarization marker changes in human monocyte derived macrophages (HMDMs) in response to M1 polarizing IFNY, M2 polarizing IL4 and IL13, AsPC-1 CM or Gli36CM measured by flow cytometry. (E) Monoclonal antibodies (mAbs) against indicated proteins reveal a specific requirement for Hsp70 in Gli36 CM to induce THP-1 differentiation. The indicated mAbs were used in THP-1 cell cultures at 200 ng in the presence of Gli36 CM equivalent to 7.4 ng py-Hsp70 (phosphotyrosine-Hsp70). (F) Increasing concentration of Hsp70 in Gli36 CM induce increases in THP-1 differentiation as measured by CD14 expression by flow cytometry. (G) Western blots of whole cell lysates from indicated cancer cell lines probed with anti-Hsp70 mAb (middle panel), pan-phosphotyrosine mAb (top panel) or anti-GAPDH antibody (lower panel). (H) Western blots of whole cell lysates from indicated cancer cell lines probed with anti-Hsp70 mAb (middle panel), anti-pt-Hsp70 (anti-phosphothreonine Hsp70; top panel) or anti-actin antibody (lower panel). (I) Western blot of CM from indicated cancer cell lines probed with pan-anti-phosphotyrosine mAb (upper panel) and anti-pt-Hsp70 mAb (lower panel). (J) Sequential immunoprecipitation from Gli36 CM by pt-Hsp70 mAb shows that all Gli36-secreted Hsp70 is phosphorylated. Gli36 CM was first immunoprecipitated with pt-Hsp70 antibody. The supernatant was subjected to a second immunoprecipitation with pt-Hsp70 and the resultant supernatant was subjected to a third immunoprecipitation using anti-Hsp70 antibody that recognizes Hsp70 irrespective of phosphorylation. The western blot shows eluates from individual immunoprecipitations probed with anti-Hsp70 mAb. (K) CD14 expression on THP-1 cells cultured in DMEM, Gli36 CM treated with alkaline phosphatase (AP) buffer or Gli36 CM treated with AP. (L) Western blots of untreated, AP treated or lambda phosphatase (LP) treated Hsp70 immunoprecipitates from Gli36 CM. Lysate indicates Gli36 total cell lysate. Western blot probed with anti-pan phosphotyrosine antibody showing py-Hsp70 (top panel) and western blot probed with anti-Hsp70 antibody showing total Hsp70 (bottom panel).

FIG. 2 shows that Py-Hsp70 is the causal factor in cancer CM induced THP-1 differentiation and is required for intra-tumor M2 macrophage maintenance. (A) Western blot showing the knockdown (KD) of Hsp70 in LLC-GFP cells (top left panel) and CD14 expression of LLC-GFP measured by flow cytometry showing reduction of THP-1 differentiation capacity of LLC-GFP CM upon Hsp70 KD (bottom left panel). Western blot showing the KD of Hsp70 in LN229 cells (top right panel) and CD14 expression of LN229 measured by flow cytometry showing the reduction of THP-1 differentiation capacity of CM upon Hsp70 KD (bottom right panel). (B) Measurement of M2 macrophage marker CD206 expression in macrophages isolated from subcutaneous tumors from WT LLC-GFP cells expressing control shRNAs, and Hsp70 KD tumors (top left and bottom left panels) and measurement of M1 macrophage marker iNOS expression in macrophages isolated from subcutaneous tumors from WT LLC-GFP cells expressing control shRNAs, and Hsp70 KD tumors (top right and bottom right panels). (C) Subcutaneous tumor growth from WT LLC-GFP cells (line 1) or Hsp70 KD LLC-GFP cells (line 2).

FIG. 3 shows that TLR2-6 is required for cancer CM-induced THP-1 differentiation and py-Hsp70 interacts with TLR2. CD14 expression of THP-1 cells cultured in CM from Gli36 (A), LLC-GFP (B), or MiaPaCa-2 (C) in the presence or the absence of TLR2 neutralizing antibodies (NAbs), TLR6 NAbs or TLR2 and TLR6 NAbs together. DMEM indicates THP-1 cells cultured in DMEM medium alone. (D) Western blot showing presence of py-Hsp70 in the TLR2 immunoprecipitates of SC cell lysates incubated with Gli36 CM. (E) CD14 expression in WT and TLR null THP-1 cells cultured in DMEM or Gli36 CM. (F) CD36 expression in WT and TLR null THP-1 cells cultured in DMEM or Gli36 CM. (G) Flow cytometric dot blots representing (E) (left panel) and (F) (right panel).

FIG. 4 shows that TLR2 is required in vivo for Gli36 CM-induced macrophage polarization and tumor growth. (A) M2 macrophage marker CD206 expression of peritoneal macrophages in WT and TLR2^{-/-} mice intraperitoneally injected with DMEM or Gli36 CM. (B) Expression of pan macrophage marker F4/80 on peritoneal macrophages in WT and TLR2 null mice intraperitoneally injected with DMEM or Gli36 CM. (C) Flow cytometric dot plots showing CD206+, CD301+M2 marker expression in peritoneal macrophages of WT and TLR2^{-/-} mice intraperitoneally injected with DMEM or Gli36 CM. (D) Tumor growth curves of LLC-GFP subcutaneous tumors in WT and TLR2^{-/-} mice. (E) Expression of CD206 in tumor macrophages isolated from LLC-GFP subcutaneous tumors in WT and TLR2^{-/-} mice analyzed by flow cytometry. (F) Representative immunohistochemistry (IHC) of tumor sections from subcutaneous LLC-GFP tumors from WT or TLR2^{-/-} mice showing Arginase 1 positive macrophages.

FIG. 5 shows the requirement of MerTK receptors for cancer CM-induced macrophage polarization in mice, py-Hsp70 association with TLR2 and MerTK and TLR2-dependent upregulation of MerTK by cancer CM with formation of a py-Hsp70-MerTK-TLR2 complex. (A) CD206 expression of macrophages in peritonea of MerTK^{-/-}, Axl^{-/-}, and WT mice injected with Gli36 CM showing the reduction of M2 polarized macrophages in peritonea of MerTK^{-/-} compared to Axl^{-/-} and WT mice, in response to Gli36 CM. (B) INOS expression of macrophages in peritonea of MerTK^{-/-}, Axl^{-/-}, and WT mice injected with Gli36 CM showing an increase in M1 polarized macrophages in peritonea of MerTK^{-/-} compared to Axl^{-/-} and WT mice in response to Gli36 CM. (C) Western blot showing the MerTK expression in WT THP-1 cells and TLR2^{-/-} THP-1 cells cultured with DMEM and Gli36 CM showing that Gli36 CM induced MerTK expression in WT THP-1 cells but not in TLR2^{-/-} THP-1 cells. (D) Western blot showing the MERTK expression in WT THP-1 cells and TLR2^{-/-} THP-1 cells cultured with DMEM and MiaPaCa-2 CM showing that MiaPaCa-2 CM induced MerTK expression in WT THP-1 cells but not in TLR2^{-/-} THP-1 cells. (E) Flow cytometric measurement of MerTK expression in WT and TLR2^{-/-} THP-1 cells in response to Gli36 CM and MiaPaCa-2 CM. (F) Immunofluorescent staining of THP-1 cells cultured in DMEM (left panel), Gli36 CM (middle panel) or MiaPaCa-2 CM (right panel) for MerTK, TLR2, and nuclei. Note strong co-localization of TLR2 and MerTK in Gli36 or MiaPaCa-2 CM treated THP-1 cells. (G) Induction of MerTK in SC cells by Gli36 CM and its inhibition by TLR2 neutralizing mAb. (H) Induction of MerTK in SC cells by Gli36 CM and its inhibition by TLR2 neutralizing mAb. (I) Immunoprecipitates of MerTK from Gli36 CM show presence of py-Hsp70 and TLR2 with western blot. (J) MerTK immunoprecipitates from medium control (DMEM) lack TLR2.

FIG. 6 shows that high surface PS cancer cell CM induces THP-1 differentiation and macrophage M2 polarization. (A) CD14 expression in THP-1 cells cultured with low surface and high surface PS cancer cell CM. (B) Representative dot plots from flow cytometry. (C) ELISA based quantified Hsp70 from CM of indicated cells. (D) Correlation between surface PS and THP-1 differentiation ability from indicated primary and cancer cell lines as measured by flow cytometric measurement of Annexin V on cancer cell lines and CD14 on CM-treated THP-1 cells. (E) Correlation between THP-1 differentiation ability and secreted Hsp70 from indicated primary and cancer cell lines, as measured by flow cytometric measurement of CD14 on CM-treated THP-1 cells and ELISA based quantification of Hsp70 from CM of indicated cells. (F) Correlation between surface PS and secreted Hsp70 from indicated primary and cancer cell lines, as measured by flow cytometric measurement of Annexin V and ELISA based quantification of Hsp70 from CM of indicated cells. (G) Western blot showing the expression of M2 markers; Arginase1 and TGM2 and M1 markers; iNOS and SOCS3 in J774 macrophages cultured with indicated low and high surface PS cancer cell lines CM. (H) Immunofluorescent microscopic analyses showing surface expression of Hsp70, surface PS and their co-localization in HPDE cells (top left panel), AsPC-1 cells (middle left panel) or MiaPaCa-2 cells (bottom left panel). The bright field image is shown to the right of each panel. (I) Bar graphs show quantification of area of exposure of surface PS (top left panel), surface Hsp70 (top right panel) and co-localization of PS and Hsp70 (bottom panel) in HPDE, AsPC-1 and MiaPaCa-2 cells. Image J software was used to calculate surface Hsp70, surface PS, colocalization and total cell area. (J) Western blot analyses of CM from indicated low and high surface PS expressing cell lines probed with anti-pt-Hsp70 mAb.

FIG. 7 shows sorted high surface PS cells from individual cancer cell lines exhibit strong THP-1 differentiation activity, macrophage M2 polarization activity and faster tumor initiation in mice. (A) Flow cytometry-based sorting of high surface PS (Annexin V high) and low surface PS (Annexin V low) cancer cells by Annexin V staining. (B-E) CD14 expression of THP-1 cells cultured with DMEM medium, sorted high surface PS cancer cells (S-High PS), sorted low surface PS cancer cells (S-Low PS) or a mix of sorted high and low surface cancer cells from AsPC-1 cells (B), LLC-GFP cells (C), cfPac1-Luc3 cells (D) and (E) Gli36 cells. (F) Mouse peritoneal M2 macrophage polarization in response to unsorted, low and high surface PS sorted LLC-GFP cells. (G) Tumor initiation in mice by high and low surface PS sorted LLC-GFP cells. The filled circles and squares show the day when the tumor was first detected in an individual mouse and the horizontal bars are the mean day of tumor initiation. (H) Model presenting proposed mechanism of immunomodulation through macrophages by cancer-secreted py-Hsp70. Cancer cells secrete py-Hsp70 through PS-rich domains on their membranes. Py-Hsp70 then binds to TLR2 receptors on macrophages and upregulates MerTK, Upregulated MerTK senses enhanced PS exposure in the TME and induces macrophage M2 polarization leading to immunosuppression and tumor growth.

FIG. 8 shows that cancer cell-secreted THP-1 differentiation activity is present in microparticle/exosome-free fraction of CM. THP-1 differentiation, as measured by flow cytometric analyses of CD14 expression, in response to DMEM, unfractionated CM, microparticle/exosome-free fraction (supernatant) of CM or microparticle/exosome fractions (pellet) from MiaPaCa-2 and Gli36 CM obtained after ultracentrifugation. (B) Monoclonal antibodies (mAbs) against indicated proteins reveal a specific requirement for Hsp70 in Gli36 CM to induce THP-1 differentiation. The indicated mAbs were used in THP-1 cell cultures at 100 ng in the presence of Gli36 CM equivalent to 1.48 ng py-Hsp70 (phosphotyrosine-Hsp70).

FIG. 9 shows that cancer cell CM generates profound morphological changes. (A) Microscopic images of THP-1 cells treated with DMEM and Gli36 CM. (B) Microscopic images of J774 cells treated with DMEM, Gli36 CM, and LLC-GFP CM and (C) Microscopic images of mouse bone marrow cells treated with M-CSF and Gli36 CM. Black arrows point to rounded morphology or flattened morphology.

FIG. 10 shows that MiaPaCa-2 CM induces macrophage marker expression in THP-1, SC and bone marrow cells. (A) Increase in CD36 expression in THP-1 cells in response to MiaPaCa-2 CM, (B) Increase in CD68 expression in THP-1 cells in response to MiaPaCa-2 CM. (C) Increase in CD68 expression in SC macrophage cell line in response to MiaPaCa-2 CM. (D) Increase in F4/80 expression in THP-1 cells in response to MiaPaCa-2 CM.

FIG. 11 shows treatment of Gli36 CM with proteinase K eliminates THP-1 differentiation activity. CD14 expression of THP-1 cells cultured with DMEM, Gli36 CM or Gli36 CM digested with proteinase K. CD14 expression was measured by flow cytometry. Gli36 CM contains 1.48 ng Hsp70.

FIG. 12 shows pancreatic and glioma tumor cells show higher Hsp70 mRNA expression than normal cells. mRNA expression of Hsp70 in pancreatic cancer (A) and glioma (B) compared to normal cells obtained from TCGA database.

FIG. 13 shows optimization of py-Hsp70 elution from Gli36 CM immunoprecipitates. (A) CD14 expression of THP-1 cells cultured with immunoprecipitated py-Hsp70 from Gli36 CM eluted with 8 M urea, 3.5 M MgCl2 or pH 2.5, pH 3, pH 3.5 and pH 4 glycine buffers. (B) Dose-dependent increase in THP-1 differentiation activity by py-Hsp70 eluted from immunoprecipitates from 10 ml and 30 ml of Gli36 CM eluted with either pH 2.5 or 3 glycine buffer results in greater CD14 expression, (top panels), and the graphical representation of THP-1 differentiation activity from eluted py-Hsp70 (bottom panel).

FIG. 14 shows surface exposure and co-localization of PS and Hsp70. Immunofluorescence microscopic analyses showing the surface expression of Hsp70, surface PS and co-localization of surface Hsp70 and PS in U87ΔEGFR cells (A) and in Gli36 cells (B). The bright field image is shown to the right of each panel.

FIG. 15 is a table (Table 1) showing cancer CM-induced secretion profile of chemokines/cytokines from THP-1 cells. Secreted cytokines and chemokines by THP-1 cells in response to Gli36 CM or MiaPaCa-2 CM. CM from untreated THP-1 cells or Gli36 CM treated THP-1 cells or Gli36 CM or MiaPaCa-2 CM treated THP-1 cells or MiaPaCa-2 were analyzed by human proteome array and specific secreted cytokines and chemokines are shown.

### DETAILED DESCRIPTION

The following description of particular embodiment(s) is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention but are presented for illustrative and descriptive purposes only.

The terminology used herein is for the purpose of describing particular embodiments only and is not intended to be limiting. As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, pH, size, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Unless otherwise defined, all terms (including technical and scientific terms) used herein have the same meaning as commonly understood by one of ordinary skill in the art to which this disclosure belongs. It will be further understood that terms such as those defined in commonly used dictionaries should be interpreted as having a meaning that is consistent with their meaning in the context of the relevant art and the present disclosure, and will not be interpreted in an idealized or overly formal sense unless expressly so defined herein.

The terms “treat,” “treatment,” and “treating,” as used herein, refer to a method of alleviating or abrogating a disease, disorder, and/or symptoms thereof in a subject.

As used herein a “subject” or “patient” refers to a mammal. Optionally, a subject or patient is a human or non-human primate. Optionally, a subject or patient is a dog, cat, horse, sheep, cow, rabbit, pig, or mouse.

An “effective amount” is defined herein in relation to the treatment of cancer as an amount of a therapeutic that will decrease, reduce, inhibit, or otherwise abrogate the progression of cancer. In some embodiments, the therapeutic agent(s) can be delivered regionally, in effective amount(s) to a particular affected region or regions of the subject's body. In some embodiments, wherein such treatment is considered more suitable, the therapeutic agent(s) can be administered systemically in effective amount(s). For example, the compound can be administered parenterally. In a specific embodiment, a therapeutic agent is delivered intravenously. In a very specific environment, the therapeutic agent is delivered directly or indirectly to a tumor microenvironment.

Impairments of the immune system are critical for development of many cancers. Cancer cells have evolved immunosuppressive mechanisms to escape host immune attack, sustain the tumor, and promote further proliferation. Reversal of the immunosuppressive tumor microenvironment (TME) is now considered a robust approach for treatment. However, the TME is a complex milieu comprised of diverse cell types that is only partially understood.

Tumor associated macrophages (TMs) and myeloid derived suppressor cells (MDSCs) are predominant mediators of robust immunosuppression in TME. Cancers elicit immunosuppression by inducing macrophage polarization into the immunosuppressive, pro-tumorigenic M2 phenotype. During tumor progression, circulating monocytes and resident macrophages are recruited to the tumor site where tumor-derived factors mediate their conversion into M2 macrophages. This phenotype is in contrast to the M1 phenotype, which is pro-inflammatory and provides anti-tumor immunity.

The development of M1 and M2 macrophages is tightly regulated and research suggests that inhibition of tumor M2 macrophage polarization is a potent therapeutic approach for treatment, as it facilitates immune activation against the tumor. At early stages of tumor development, both M1 anti-tumor and M2 pro-tumor TMs are present in the TME. However, as tumors progress, the TME is predominantly composed of M2 polarized TMs, with a decline in M1 polarized TMs.

A candidate factor increasingly appreciated to potentially mediate the actions of cancer cells on the TME is Heat shock protein-70 (Hsp70). Hsp70 is overexpressed in a variety of cancers, plays critical roles in tumor growth, and is known to be released by cancer cells. Depletion of Hsp70 was shown to reduce tumor growth in pancreatic ductal adenocarcinoma, glioblastoma, colon, prostate and hepatocellular carcinomas. Extracellular Hsp70 has been reported in tumors and serum Hsp70 is increased in patients with glioblastoma, pancreatic cancer, and lung cancer. The secretion mechanism for Hsp70 is uncertain but evidence suggests externalization occurs via a non-conventional mode of secretion in association with membrane rafts, association with other proteins that contain a signal domain, and/or based on an association with phosphatidylserine (PS).

Phosphatidylserine is known to interact with Hsp70. Non-apoptotic cancer cells expose elevated levels of PS on their surface compared to normal cells, where PS is predominantly localized to the inner leaflet of the plasma membrane. Tumor cells vary in surface exposure of PS and cancer cells with high surface PS exhibit reduced flippase activity (an enzyme involved in PS translocation from outer leaflet to inner leaflet), increased intracellular calcium, and high total cellular PS compared to low surface PS cancer cells and healthy cells. Externalized PS normally serves as a signal for dying cells to be cleared by macrophages. PS receptors are expressed on immune cells and recent studies have shown immunoregulatory functions for PS. Macrophages recognize PS on apoptotic cells via PS receptors to trigger phagocytosis.

Unlike intracellular Hsp70, the functions of secreted Hsp70 are unclear. Even though Hsp70 has been shown to be hyper phosphorylated in cancer, a requirement of phosphorylation for Hsp70 secretion and function remains to be determined. An intriguing extracellular signaling pathway are the Toll-like receptors (TLRs), a family of receptors well-documented to exert actions on immune function. TLRs have been reported to bind to Hsp70 triggering innate immunity and to exert specific immunomodulator actions (including on macrophages) in the TME. TLRs are known for their pro-inflammatory function. Binding of agonists mimicking bacterial structures to TLR2 is known to induce tumor suppression. Macrophages express TLRs and, upon recognition of bacterial or pathogen structures, trigger an inflammatory response. However, a role for macrophage TLRs in the regulation of tumor induced immunosuppression is not known. In addition to TLRs and receptor tyrosine kinases consisting Tyro3, Axl, and Mer receptor tyrosine kinases (TKs) play a critical role in macrophage M2 polarization in tumor TME, as evident by inhibition of tumor M2 macrophage polarization in Tyro3, Axl and MerTK family receptor knockout in mouse cancer models.

The present disclosure evidences that a predominant activity in cancer conditioned media can be accounted for by secreted Hsp70 that is specifically tyrosine phosphorylated (py-Hsp70). Py-Hsp70 was found to interact with TLR2 followed by upregulation of MerTK, suggesting a mechanism in which TLR2 triggers the MerTK receptor linked directly with macrophage polarization and immunosuppression. Total Hsp70 knockdown or lack of TLR2 led to decreased tumor growth and reduction in intra-tumor M2 polarized macrophages. The discovery of cancer-secreted py-Hsp70 induced TLR2-dependent induction of MerTK opens up a new amplification mechanism for tumor immunosuppression and a therapeutic target for cancer treatment by facilitating immune reactivation. Further, secreted Hsp70 serves as a marker for identification of cancers susceptible to PS-targeted therapies.

The present disclosure identifies a novel immunosuppressive signaling mechanism between cancer cells and macrophages, wherein, Hsp70, located in association with PS on cancer cells and secreted in greater abundance by high PS cancer cells triggers immunosuppression through TLR2 and MerTK receptors. The secreted Hsp70 is tyrosine phosphorylated in contrast to intracellular Hsp70 and acts through macrophage TLR2, assembles with MerTK and induces upregulation of MerTK receptors, in a TLR2 dependent manner. Non-phosphorylated Hsp70 was not detected in the cancer-conditioned medium and enzymatic removal of phosphorylation led to loss of macrophage differentiation, indicating the importance of Hsp70 phosphorylation in the immunosuppression process. Furthermore, the data supports a requirement of py-Hsp70, MerTK, and TLR2 in intra-tumor macrophage M2 polarization and tumor growth, as evident by impaired macrophage polarization and impaired tumor growth both in Hsp70 knockdown tumors and in tumors of TLR2 null mice. These findings reveal a tumor immunosuppression mechanism that involves MerTK amplification triggered by py-Hsp70 through TLR2 and identify py-Hsp70 as a potent therapeutic target to prevent tumor immunosuppression and marker of susceptibility to PS-targeted therapeutics.

Cancer-secreted py-Hsp70 is a potent regulator of immunosuppressive M2 polarization. Immune-neutralization experiments revealed that Hsp70 is the critical protein with potent macrophage differentiation ability, as anti-Hsp70 mAbs potently inhibited cancer CM induced macrophage differentiation compared to partial inhibition of macrophage differentiation by antibodies against PAI1, CYR 61, and Hsp90.

Hsps were originally described as chaperone proteins implicated in protein folding required for stress response after temperature elevation and other proteotoxic stresses, to prevent damage of cellular structures and thereby protect essential cellular functions. It is well known that Hsp70 is overexpressed in a variety of cancers and play critical role in tumor growth, as evident by reduced tumor growth upon Hsp70 knockdown in tumor cells. An increase in serum Hsp70 levels has been reported in glioblastoma and pancreatic cancers. However, a function for secreted Hsp70 in immunosuppression is not known. Thus, the finding that cancer-secreted py-Hsp70 controls macrophage polarization through TLR2 and MerTK receptors identifies molecular communication between cancer cells and macrophages that has potential of therapeutic targeting.

Even though intracellular Hsp70 in cancer is known to be hyper phosphorylated, the phosphorylation status of secreted Hsp70 was previously unknown. The phosphorylation of Hsp70 enables increased binding to co-chaperones and Hsp70 phosphorylation has been linked to increased cellular proliferation. Surprisingly, it has been found that intracellular Hsp70 lacks tyrosine phosphorylation but cancer-secreted Hsp70 is tyrosine phosphorylated.

Another important aspect of this study is the utilization by cancers of the macrophage TLR2 receptor by py-Hsp70. Although TLR family of receptors are mostly known for pro-inflammatory functions, this disclosure identifies the usage of TLR2 receptor by cancer py-Hsp70 to trigger anti-inflammatory macrophage M2 polarization through induction of MerTK receptors on macrophages. The requirement of TLR2 for cancer CM macrophage differentiation function was underscored by failure of differentiation in TLR2 null THP-1 cells and reduction in M2 macrophages in peritonea of TLR2 null mice in response to cancer CM. Highlighting the importance of these molecules in macrophage M2 polarization, it was found that targeting either TLR2, MerTK or Hsp70 reduced M2 polarized macrophages, as shown within the tumors established from Hsp70 knockdown cells, in tumors of TLR2 null mice and in peritoneal macrophages of MerTK null mice.

Although MerTK receptors are known to be involved in macrophage M2 polarization, collaboration between TLR2 and MerTK receptors in the induction of immunosuppression was not previously known. The present disclosure evidences coordination between TLR2 and MerTK receptors on macrophages, wherein TLR2 receptors induce MerTK receptors and associate together with Hsp70.

The present investigators have found larger areas of surface co-localization of Hsp70 and PS in high surface PS cancer cell lines compared to low surface PS cancer cells, indicating a PS-associated externalization of Hsp70 in cancer cells. A correlation between cancer cell surface PS, secreted py-Hsp70, and macrophage differentiation has been demonstrated. In particular it has been found that the higher PS cellular fraction in individual cancer types, the higher the macrophage differentiation ability and the faster the tumor initiation ability, which is of therapeutic interest to target these higher surface PS cells in individual cancer types to inhibit cancer immunosuppression.

Together, the data indicate that cancers secrete py-Hsp70, in a PS-associated manner, and that the secreted py-Hsp70 acts on macrophages through TLR2 and promotes MerTK induction to facilitate amplification of M2 polarization. Detection of cancer-secreted soluble Hsp70, and in particular py-Hsp70, represents a new marker for identifying high surface PS cancer cells that would be sensitive to PS-targeted therapies.

In one embodiment, a method of non-invasively diagnosing and treating a cancer characterized by high surface phosphatidylserine (PS) expression in a subject in need thereof is provided, the method comprising: obtaining a liquid biological sample from the subject; detecting a presence of cancer-secreted soluble phosphorylated Heat shock protein-70 (Hsp70) in the liquid biological sample; diagnosing the subject with cancer characterized by high surface PS expression when cancer-secreted soluble phosphorylated Hsp70 is present in the liquid biological sample; and administering an anti-cancer therapy targeted to high surface PS expressing-cancers to the diagnosed subject. In a specific embodiment, the secreted phosphorylated Hsp70 detected in the biological sample is tyrosine-phosphorylated Hsp70 (py-Hsp70).

Various liquid biological samples obtained from the subject are suitable for use in the instant methods. In embodiments, suitable liquid biological fluids include blood, serum, plasma, urine, breastmilk, saliva, tears, sweat, gastrointestinal secretions, homogenates of tissues or tumors, synovial fluid, feces, sputum, cyst fluid, amniotic fluid, cerebrospinal fluid, peritoneal fluid, lung lavage fluid, semen, lymphatic fluid, prostatic fluid, and the like. In a specific embodiment, the liquid biological sample is selected from the group consisting of blood, serum, plasma, urine, breastmilk, saliva, tears, and sweat. In a very specific embodiment, the liquid biological fluid is selected from the group consisting of blood, serum, plasma, urine, and breastmilk.

Different types of cancer cells vary in the level of surface PS expressed on the outer surface of the lipid bilayer of the cell. While cancer cells typically comprise elevated levels of surface PS compared to non-cancerous cells, levels of surface PS may vary between cancers and between cancer patients. Cancer cells that express relatively higher levels of surface PS are more likely to be sensitive to treatment with anti-cancer therapeutics targeted to PS. In embodiments, cells characterized as having high levels of surface PS are cells having an Annexin V fluorescence of greater than or equal to about 2000 mean fluorescence intensity (WI). In embodiments, cells characterized as having low levels of surface PS are cells having an Annexin V fluorescence of less than or equal to about 1500 MFI. In embodiments, MFI is measured by flow cytometry.

In embodiments, cancer cells characterized by high surface PS are cells having an Annexin V fluorescence of greater than or equal to about 2000, about 2500, about 3000, about 3500, about 4000, or about 4500 mean fluorescence intensity (MFI).

In embodiments, cancer cells characterized by low surface PS are cells having an Annexin V fluorescence of less than about 2000, about 1500, about 1250, or about 1000 MFI.

Various types of cancer are characterized as having high or relatively higher levels of surface PS. In embodiments, cancers characterized by high surface PS expression include, but are not limited to, pancreatic cancer, glioma, melanoma, lung cancer, colorectal cancer, and pediatric cancers such as neuroblastoma, malignant peripheral nerve sheath tumors (MPNST), and diffuse intrinsic pontine glioma (DIPG). However, the skilled artisan will appreciate that any cancer having surface PS expression as set forth herein is considered to be a cancer predicted to be sensitive to PS-targeted anti-cancer therapies.

Anti-cancer therapies targeted to high surface PS expressing-cancers include, but are not limited to, bavituximab, Saposin C-dioleoylphosphatidylserine (SapC-DOPS, also known as BXQ-350), phosphatidylcholine-stearylamine (PC-SA), DPA-CY3 1-palmitoyl-2-oleoyl-sn-glycero-3-phosphocholine (DPA-CY3/POPC), Chalepin, human annexin-V, PGN634, mch1N11, PPS1, PPS1D1, PSBP-6, hexapeptide E3, TSR-022, MBG543, BMS-986258, LY3321367, Sitravatinib, R428, TP0903, BMS-777607, NPS1034, MRX-2843, UNC2025, UNC3133, ONO-7475, Tyro3 inhibitors, and 5F9. Additional therapeutic agents include lactadherin-like and annexin-like compounds. Various PS-targeted therapies are disclosed in Chang, et al., Targeting phosphatidylserine for Cancer therapy: prospects and challenges, Theranostics 10(20): 9214-29 (2020).

Optionally, the methods disclosed herein further comprise detecting the presence of one or more additional biomarkers in the liquid biological sample. In embodiments, the one or more additional biomarkers is selected from the group consisting of Heat shock protein-90 (Hsp90), moesin, S5a, polyubiquitin-B, ubiquitin-60S ribosomal protein L40, beta-hexosaminidase, neuronal pentraxin-1, variant surface antigen D, fibulin-1, nidogen-1, alpha enolase, Cyr61, PAI1, EF1 alpha, IGFBP3, versican, sulfydryl oxidase, and combinations thereof. In embodiments, presence of the one or more additional markers in the liquid biological sample, in combination with presence of py-Hsp70, is indicative of a cancer having high surface PS-expression, suitable for treatment with PS-targeting therapies.

In embodiments, the methods of diagnosing and treating cancer disclosed herein further comprise administering to the subject one or more additional anti-cancer therapeutics selected from the group consisting of an Hsp70 inhibitor, a Toll-like receptor 2 (TLR2) inhibitor, and a Mer tyrosine kinase (MerTK) inhibitor.

Various Hsp70 inhibitors are known in the art or are currently under development and suitable for use in the disclosed methods. In embodiments, the Hsp70 inhibitor selected from the group consisting of apoptozole, JG-13, JG-98, MAL3-101, MKT-077, spergualin, YM-01, YM-08, methylene blue, and combinations thereof. In embodiments, the Hsp70 inhibitor is a selective Hsp70 inhibitor. Exemplary Hsp70 inhibitors are disclosed in US 2011/0160160, US 2020/0237860, and at www.hsp70.com/inhibitors/, last accessed Apr. 1, 2021. In a specific embodiment, the Hsp70 inhibitor inactivates py-Hsp70. In a very specific embodiment, the phosphorylated Hsp70 inhibitor is saposin C dioleoylphosphatidylglycerol (SapC-DOPG).

Various TLR2 inhibitors suitable for use in the disclosed methods are currently known in the art or under development. In embodiments, TLR2 inhibitors are selected from the group consisting of C29, ortho-vanillin, AT5, CUCPT-22, MMG-11, T2.5, and OPN-305, and combinations thereof.

In embodiments, MerTK inhibitors suitable for use in the disclosed methods are selected from the group consisting of UNC569, UNC1062, UNC4203, UNC2025, UNC2250, MRX-2843, ONO-7475, RXDX-106, S49076, merestinib, and combinations there. Additional MerTK inhibitors are disclosed in US 2018/0002444, US 2017/0165261, and Zhao, et al., Highly selective MERTK inhibitors achieved by a single methyl group, J. Med. Chem. 61(22): 10242-54 (2018), Branchford, et al., The small-molecule MERTK inhibitor UNC2025 decreases platelet activation and prevents thrombosis, J. Thromb. Haemost. 16(2): 352-63 (2018), Koda, et al., Effects of MERTK Inhibitors UNC569 and UNC 1062 on the Growth of Acute Myeloid Leukaemia Cells, Anticancer Research 38: 199-204 (2018).

In embodiments, the anti-cancer therapeutics disclosed herein may be administered intravenously, parenterally, orally, topically, or regionally to the subject. In another embodiment, the anti-cancer therapeutic is administered directly or indirectly to the tumor microenvironment.

In some aspects, the methods of the present disclosure further include administering an additional chemotherapeutic to the cell. In embodiments, the one or more additional anti-cancer agents is selected from the group consisting of chemotherapeutic agents, radiotherapeutic agents, cytokines, anti-angiogenic agents, apoptosis-inducing agents, and anti-cancer immunotoxins. By way of example, such additional chemotherapeutic agents may include one or more of everolimus, erlotinib, 5-fluorouracil, irinotrecan, olaparib, mitomycin, paclitaxel, sunitinib, FOLFIRINOX, cisplatin, oxaliplatin, lanreotide, lutetium Lu 177-dotatate, bevacizumab, carmustine, naxitamab, lomustine, temozolomide, afatinib, alectinib, pemetrexed, brigatinib, atezolizumab, capmatinib, carboplatin, cemoplimab, ceritinib, crizotinib, ramucirumab, dabrafenib, docetaxel, doxorubicin, durvalumab, entrectinib, pralsetinib, gefitinib, gemcitabine, ipilimumab, pembrolizumab, lorlatinib, trametinib, methotrexate, necitumumab, nivolumab, osimertinib, selpercatinib, tepotinib, trametinib, vinorelbine, or combinations thereof. In some aspects, the additional therapy may be one or more of an antibody therapy, a gene silencing therapy, a vaccine therapy, or a radiation therapy. The additional anti-cancer agent may be administered simultaneously with the PS-targeted therapy, sequentially with the PS-targeted therapy, and/or asynchronously with the PS-targeted therapy.

In another embodiment, a method is provided for monitoring the therapeutic efficacy of a treatment in a subject with a cancer characterized by high surface phosphatidylserine (PS) expression, the method comprising: treating the subject with an anti-cancer therapy targeted to high surface PS expressing-cancers; measuring the level of cancer-secreted soluble tyrosine-phosphorylated Hsp70 (py-Hsp70) in a liquid biological sample obtained from the subject after administering the anti-cancer therapy; and altering the dosage of the anti-cancer therapy, the frequency of dosing the anti-cancer therapy, or the course of therapy administered to the subject based on the level of cancer-secreted soluble py-Hsp70 measured.

It is within the purview of the ordinary skilled artisan to make adjustments to the anti-cancer therapy targeted to high surface PS expressing-cancers based on the level of py-Hsp measured in the biological sample. Optionally, the method further comprises obtaining a baseline measurement of py-Hsp levels in a liquid biological sample from the patient prior to commencing therapy. After therapy, the clinician may alter the dose, dose frequency, or choice of therapeutic agent based on how well the cancer has responded to therapy. For example, if the level of py-Hsp70 increases or is unchanged after therapy, the result may indicate that the tumor microenvironment continues to be populated with cells expressing surface PS, such that a higher dose, more frequent dosing, or a change in course of therapy may be warranted to effectively treat the cancer. If, for example, the level of py-Hsp decreases after therapy, the result may indicate that the tumor microenvironment is characterized by a reduction in surface PS-expression, such that the clinician may conclude that the selected therapy is effectively treating the cancer and that no change in dose, frequency, or course of therapy is warranted.

Various methods for measuring a level of cancer-secreted soluble tyrosine-phosphorylated Hsp70 in a liquid biological sample are known in the art. In embodiments, soluble py-Hsp70 levels are measured by ELISA, Western blot assay, HPLC, LC-MS, or other suitable technique.

In embodiments, the anti-cancer therapy targeted to high surface PS expressing-cancers is selected from the group consisting of bavituximab, Saposin C-dioleoylphosphatidylserine (SapC-DOPS), phosphatidylcholine-stearylamine (PC-SA), DPA-CY3 1-palmitoyl-2-oleoyl-sn-glycero-3-phosphocholine (DPA-CY3/POPC), Chalepin, human annexin-V, PGN634, mch1N11, PPS1, PPS1D1, PSBP-6, hexapeptide E3, TSR-022, MBG543, BMS-986258, LY3321367, Sitravatinib, R428, TP0903, BMS-777607, NPS1034, MRX-2843, UNC2025, UNC3133, ONO-7475, Tyro3 inhibitors, 5F9, and combinations thereof.

In embodiments, the method for monitoring further comprises administering to the subject one or more additional anti-cancer therapeutics. Any of the anti-cancer therapeutics disclosed herein is suitable for use. In a specific embodiment, the anti-cancer therapeutic is selected from an Hsp70 inhibitor, a Toll-like receptor 2 (TLR2) inhibitor, and a Mer tyrosine kinase (MerTK) inhibitor and is administered in combination with the one or more anti-cancer therapies targeted to high surface PS expressing-cancers.

In embodiments of the methods according to the present disclosure, high surface PS-expressing cancer cells are cells having an Annexin V fluorescence of greater than or equal to about 2000 MFI; and low surface PS-expressing cancer cells are cells having an Annexin V fluorescence of less than or equal to about 1500 MFI.

Aspects of the present disclosure can be described with reference to the following numbered clauses, with preferred features laid out in dependent clauses.
1. A method of non-invasively diagnosing and treating a cancer characterized by high surface phosphatidylserine (PS) expression in a subject in need thereof, the method comprising: obtaining a liquid biological sample from the subject; detecting a presence of cancer-secreted soluble phosphorylated Heat shock protein-70 (Hsp70) in the liquid biological sample; diagnosing the subject with the cancer characterized by high surface PS expression when cancer-secreted soluble phosphorylated Hsp70 is present in the liquid biological sample; and administering an anti-cancer therapy targeted to high surface PS expressing-cancers to the diagnosed subject.
2. The method according to clause 1, wherein the liquid biological sample is selected from the group consisting of blood, serum, plasma, urine, breastmilk, saliva, tears, and sweat.
3. The method according to any of the preceding clauses, wherein the secreted phosphorylated Hsp70 is tyrosine-phosphorylated Hsp70 (py-Hsp70).
4. The method according to any of the preceding clauses, wherein the cancer characterized by high surface PS expression is selected from the group consisting of pancreatic cancer, glioma, melanoma, lung cancer, colorectal cancer, and pediatric cancers.
5. The method according to any of the preceding clauses, wherein the anti-cancer therapy targeted to high surface PS expressing-cancers is selected from the group consisting of bavituximab, Saposin C-dioleoylphosphatidylserine (SapC-DOPS), phosphatidylcholine-stearylamine (PC-SA), DPA-CY3 1-palmitoyl-2-oleoyl-sn-glycero-3-phosphocholine (DPA-CY3/POPC), Chalepin, human annexin-V, PGN634, mch1N11, PPS1, PPS1D1, PSBP-6, hexapeptide E3, TSR-022, MBG543, BMS-986258, LY3321367, Sitravatinib, R428, TP0903, BMS-777607, NPS1034, MRX-2843, UNC2025, UNC3133, ONO-7475, Tyro3 inhibitors, 5F9, and combinations thereof
6. The method according to any of the preceding clauses, wherein the method further comprises detecting presence of one or more additional biomarkers in the liquid biological sample.
7. The method according to clause 6, wherein the one or more additional biomarkers is selected from the group consisting of Heat shock protein-90 (Hsp90), moesin, S5a, polyubiquitin-B, ubiquitin-60S ribosomal protein L40, beta-hexosaminidase, neuronal pentraxin-1, variant surface antigen D, fibulin-1, nidogen-1, alpha enolase, Cyr61, PAI1, EF1 alpha, IGFBP3, versican, and sulfydryl oxidase.
8. The method according to any of the preceding clauses, further comprising administering to the subject one or more additional anti-cancer therapeutics selected from the group consisting of an Hsp70 inhibitor, a Toll-like receptor 2 (TLR2) inhibitor, and a Mer tyrosine kinase (MerTK) inhibitor.
9. The method according to clause 8, wherein the Hsp70 inhibitor is selected from the group consisting of apoptozole, JG-13, JG-98, MAL3-101, MKT-077, spergualin, YM-01, YM-08, methylene blue, and combinations thereof.
10. The method according to clause 8, wherein the TLR2 inhibitor is selected from the group consisting of C29, ortho-vanillin, AT5, CUCPT-22, MMG-11, T2.5, OPN-305, and combinations thereof.
11. The method according to clause 8, wherein the MerTK inhibitor is selected from the group consisting of UNC569, UNC1062, UNC4203, UNC2025, UNC2250, MRX-2843, ONO-7475, RXDX-106, S49076, merestinib, and combinations thereof.
12. The method according to clause 8, wherein the Hsp70 inhibitor inactivates py-Hsp70.
13. The method according to clause 12, wherein the Hsp70 inhibitor is SapC-DOPG.
14. The method according to any of the preceding clauses, wherein administering comprising administering to the tumor microenvironment.
15. The method according to any of the preceding clauses, further comprising administering to the subject one or more additional anti-cancer agents selected from the group consisting of chemotherapeutic agents, radiotherapeutic agents, cytokines, anti-angiogenic agents, apoptosis-inducing agents, and anti-cancer immunotoxins.
16. A method for monitoring therapeutic efficacy of a treatment in a subject with a cancer characterized by high surface phosphatidylserine (PS) expression, the method comprising: treating the subject with an anti-cancer therapy targeted to high surface PS expressing-cancers; measuring a level of cancer-secreted soluble tyrosine-phosphorylated Hsp70 (py-Hsp70) in a liquid biological sample obtained from the subject after administering the anti-cancer therapy; and altering dosage of the anti-cancer therapy, frequency of dosing the anti-cancer therapy, or course of therapy administered to the subject based on the level of cancer-secreted soluble py-Hsp70 measured.
17. The method according to clause 16, wherein the anti-cancer therapy targeted to high surface PS expressing-cancers is selected from the group consisting of bavituximab, Saposin C-dioleoylphosphatidylserine (SapC-DOPS), phosphatidylcholine-stearylamine (PC-SA), DPA-CY3 1-palmitoyl-2-oleoyl-sn-glycero-3-phosphocholine (DPA-CY3/POPC), Chalepin, human annexin-V, PGN634, mch1N11, PPS1, PPS1D1, PSBP-6, hexapeptide E3, TSR-022, MBG543, BMS-986258, LY3321367, Sitravatinib, R428, TP0903, BMS-777607, NPS1034, MRX-2843, UNC2025, UNC3133, ONO-7475, Tyro3 inhibitors, 5F9, and combinations thereof
18. The method according to any of clauses 16-17, wherein the liquid biological sample is selected from the group consisting of blood, serum, plasma, urine, breastmilk, saliva, tears, and sweat.
19. The method according to any of clauses 16-18, wherein the cancer characterized by high surface PS expression is selected from the group consisting of pancreatic cancer, glioma, melanoma, lung cancer, colorectal cancer, and pediatric cancers.
20. The method according to any of clauses 16-19, further comprising administering to the subject one or more additional anti-cancer therapeutics selected from the group consisting of an Hsp70 inhibitor, a Toll-like receptor 2 (TLR2) inhibitor, and a Mer tyrosine kinase (MerTK) inhibitor, in combination with the one or more anti-cancer therapies targeted to high surface PS expressing-cancers.
21. The method according to any of the preceding clauses, wherein high surface PS-expressing cancer cells are cells having an Annexin V fluorescence of greater than or equal to about 2000 MFI; and wherein low surface PS-expressing cancer cells are cells having an Annexin V fluorescence of less than or equal to about 1500 MFI.

### EXAMPLES

### Example 1: Materials and Methods

### Cell Culture

MDA MB 231 were grown in RPMI (Fisher Scientific). WT/TLR2 null THP-1 cells, human SC macrophage cells and mouse J774 macrophage cells were cultured in RPMI with 25 mM HEPES. All the other cell lines were cultured in DMEM and all media were supplemented with 10% FBS and 1% Penicillin/Streptomycin. Human Astrocytes were cultured in Astrocyte Cell Medium (ScienCell) supplemented with the provided growth factor supplements, FBS and antibiotics. All cells were cultured in a 5% CO₂ incubator at 37° C. Cells were routinely tested for mycoplasma contamination. No cross-contamination was observed in the cell lines, as determined by cellular morphology and growth parameters. Authentication of cell lines was not conducted by the authors.

### Generation of Mouse Bone Marrow-Derived Macrophages (BMDM)

Mouse bone marrow was obtained from tibias and femurs of C57BL/6J mice (7-8 weeks old). After erythrocyte lysis, BMDMs were generated by growing cells in RPMI, containing mouse MCSF (PeproTech, Rocky Hill, NJ) for 10 days with fresh cytokine supplemented medium every three days.

### Generation of Human Monocyte-Derived Macrophages (HMDM)

Human peripheral blood monocytes were purchased from ZenBio (Durham, NC). HMDMs were generated by culturing monocytes in RPMI supplemented with human MCSF (PeproTech) for 10 days. Fresh media and cytokines were added every three days.

### Macrophage Polarization of BMDM and HMDM

BMDMs were polarized into M2 macrophages by culturing with mouse Il-4 and Il-10 (PeproTech; 10 ng/ml) for 3 days. HMDMs were polarized into the M2 phenotype by culture with human Il-4 and Il-13 (PeproTech; 10 ng/ml) for 3 days. M1 polarization was induced by culture of BMDMs and HMDMs with mouse and human IFN-γ respectively (PeproTech). Alternatively, BMDM and HMDM were cultured with conditioned media (CM) obtained from human cancer cells.

Generation of Serum-Free, Exosome/Microparticle Free Conditioned Media (CM) from Human/Mouse Cancer Cell Lines and THP-1 Differentiation Assay

Human/mouse cancer cell lines were grown in their respective media until 70% confluency in 10 cm Corning tissue culture plates (ThermoFisher, Waltham, MA), at which time, the media was removed. The cells were washed twice with serum-free media, to remove remnants of serum and dead cells and replenished with serum-free media. After 24 hrs, CM was collected, centrifuged at 10,000 g to remove cellular debris, followed by ultracentrifugation at 100,000×g to remove extracellular exosomes and microparticles. THP-1 cells (2×10⁵) were cultured in 1 ml of CM normalized to 100 μg of total cellular protein from indicated cancer cell lines. Control THP-1 cells were grown in DMEM. After 24 hrs, control and CM-treated cells were centrifuged then incubated with CD14-PE conjugated antibody (eBioscience, San Diego, CA) and propidium iodide (PI; BD Biosciences, Franklin Lakes, NJ) in 100 μl FACS buffer for 30 minutes on ice. Cells were washed with flow cytometry buffer (PBS+2% FBS) and CD14 expression measured by flow cytometry. In studies involving assessment of microparticles/exosomes in THP-1 differentiation, THP-1 cells were cultured in unfractionated CM, CM devoid of microparticles/exosomes or with the microparticle/exosome fraction of the CM for 24 hrs and differentiation was measured by flow cytometric assessment of CD14 expression as described above.

### THP-1 Culture in the Presence of Neutralizing Antibodies

For neutralization studies involving TLRs THP-1 cells were cultured with CM obtained from human cancer cell lines, in the presence or absence of neutralizing antibodies (eBioscience; 1 μg/ml) against TLR2, TLR6 and control IgG. After 24 hrs, cells were harvested, stained with PI to exclude dead cells, and CD14, THP-1 differentiation was analyzed as above. For neutralization studies involving py-Hsp70, Enolase, Moesin, SSA, and control IgG THP-1 cells were incubated with 200 ng of antibody (Santa Cruz Biotechnology, Dallas, TX) in 1 ml of culture medium for 24 hrs, after which the cells were harvested by centrifugation stained with PI to exclude dead cells and CD14, THP-1 differentiation was analyzed as above.

### Human Cytokine/Chemokine Array

Cytokine/chemokine profiles from Gli36 or MiaPaCa-2 CM, 24 hr. THP-1 culture supernatants or THP-1 culture supernatants after 24 hr. treatment with Gli36 CM or MiaPaCa-2 CM or were obtained using the Proteome Profiler Human Cytokine Array kit (R&D Systems, Minneapolis, MN) as instructed by the manufacturer. Briefly, cytokine/chemokine array panel membranes spotted with specific antibodies against cytokines/chemokines were incubated with 2 ml of the above indicated culture supernatants, followed by incubation with detection antibodies then the membranes were developed with chemiluminescence to detect cytokines/chemokines.

### Flow Cytometry

THP-1, J774, BMDMs, HMDMs or tumor derived macrophages were washed once with PBS, centrifuged and cell pellets were incubated on ice with respective antibodies in flow cytometry buffer (PBS with 2% FBS). After a 45 min. incubation, cells were washed twice with flow cytometry buffer and examined. THP-1 differentiation was assessed by flow cytometric measurement of PE-conjugated CD14 (eBioscience). For mouse M2 polarization, either M2 polarized BMDM or CM treated BMDM were stained with mouse F4/80 (for the total macrophage population; eBioscience) and mouse M2 specific markers CD206-APC, CD301-FITC (BioLegend, San Diego, CA). For Human HMDM M2 polarization, cells were stained with CD163-FITC, CD206-APC or CD14-PE (eBioscience). PI was added to all stained cells to gate out dead cells. Stained cells were analyzed with a BD Fortessa flow cytometer.

### Intracellular Staining of Cells for Flow Cytometry Analyses

Cells were washed twice with PBS and fixed using 2% formaldehyde for 30 mins. After fixation, cells were spun down and washed two times with PBS and pelleted. Cell pellets were incubated with permeabilization buffer (eBioscience) for 30 mins, followed by centrifugation. These cell pellets were incubated for 1 hr. with antibodies against intracellular antigens in permeabilization buffer. After two washes with permeabilization buffer and one with flow cytometry buffer the cells were analyzed.

Injection of Conditioned Media into WT, TLR2^{-/-}, MerTK^{-/-}, Axl^{-/-}, and MerTK^{-/-}, Axl^{-/-} Mice and Analyses of Peritoneal Macrophages

WT and TLR2-deficient C57BL/6J male mice, 6 weeks old were purchased from Jackson Laboratories (Bar Harbor, ME). MerTK^{-/-}, Axl^{-/-} and MerTK^{-/-}/Axl^{-/-} mice were generated by us. One milliliter of Gli36 CM was injected intraperitoneally twice with a gap of 5 hrs. Peritoneal macrophages were collected 24 hrs later and stained with PI and F4/80. Expression of CD206 and iNOS was analyzed by flow cytometry.

### Lentiviral-Mediated Knockdown of Hsp70

The pLKO.1 vectors expressing shRNAs targeting Hsp70 were obtained from Sigma-Aldrich (St. Louis, MO). The pLKO.1 lentiviruses were packaged in HEK-293T cells by co-transfecting the pMD2.G (VSV G) envelope plasmid and the Gag, Pol expressing psPAX2 packaging plasmid. These cells were cultured for 48 hrs after transfection and the lentiviral particles were collected from the supernatants and used to transduce LLC and LN229 cells. Thirty-six hrs post infection, gene silencing efficiency was analyzed by immunoblotting for the Hsp70 protein.

### Quantification of Cancer CM Hsp70 by ELISA

Human/mouse cancer cell lines were grown in their respective media until 70% confluency in 10 cm Corning tissue culture plates (ThermoFisher, Waltham, MA). After removing the media, the cells were washed twice with serum-free media to remove remnants of serum and dead cells and replenished with serum-free media. After 24 hrs, CM was collected by ultracentrifugation at 100,000×g to remove extracellular exosomes and microparticles. Hsp70 was quantified in CM using an Hsp70 ELISA Kit (ThermoFisher, Waltham, MA) according to manufacturer's protocol.

### Immunoprecipitation

Serial immunoprecipitations were performed using Gli36 CM and anti-phosphothreonine Hsp70 specific mAb (pt-Hsp70) (39) and anti-Hsp70 mAb (Santa Cruz Biotechnology). Essentially, Gli36 CM was first immunoprecipitated with anti-pt-Hsp70. The supernatant after the first immunoprecipitation was further subjected to two rounds of immunoprecipitation with anti-pt-Hsp70. The supernatant after three rounds of depletion of pt-Hsp70 was used for immunoprecipitation with anti-Hsp70 mAb which recognizes Hsp70 irrespective of phosphorylation status. All the immunoprecipitates were analyzed by western blotting and probed for presence of Hsp70. To determine whether MerTK, TLR2 and Hsp70 exist in a complex after cancer CM treatment of SC macrophage cells or THP-1 cells, cells were first treated with indicated cancer cell CM and MerTK was immunoprecipitated using anti-MerTK mAb (Santa Cruz Biotechnology) and incubated with 10× concentrated Gli36 CM (obtained by passing Gli36 CM through 10 kDa molecular weight cutoff filter) overnight at 4° C., followed by addition of protein AG beads. As a control, SC macrophage cells or THP-1 cells are treated with DMEM medium and MerTK was immunoprecipitated using anti-MerTK mAb followed by further incubation with DMEM medium overnight at 4° C., followed by addition of protein AG beads. After addition of Protein AG beads, the mixture was incubated at 4° C. The beads were centrifuged and washed twice with cell lysis buffer then suspended in SDS loading buffer and analyzed by western blot for Hsp70, TLR2, and MerTK.

### Affinity Purification of Py-Hsp70 and Assessment of THP-1 Differentiation Activity

10, 20, and 30 ml of Gli36 CM was concentrated to 1 ml, incubated with anti-Hsp70 specific mAb overnight at 4° C., followed by addition of protein AG beads then again incubated overnight at 4° C. Afterwards, the beads were pelleted by centrifugation and washed 2 times with PBS. Bound proteins were eluted by using Tris (20 mM)-glycine (200 mM) buffer (pH 2.5, 3.0, 3.5 or 4.0). Eluates were neutralized by addition of an equal volume of pH 8.5 neutralization buffer. Alternatively, the beads were eluted with 3.5 MgCl₂ and the eluates were renatured by dialysis against PBS using a 10 kDa cut off dialysis membrane. In addition, proteins were removed from the antibodies using 8 M urea and the eluates were serially dialyzed against buffers containing 6, 4, and 2 M urea and finally against PBS. Eluates 100 μl together with 1 ml DMEM medium are added to THP-1 cells and after 1 day THP-1 differentiation was measured by CD14 expression analyses by flow cytometry.

### Dephosphorylation of Cancer Cell Conditioned Media (CM)

Gli36 CM was concentrated using a 10 kDa molecular weight cut off membrane.

Concentrated CM was immunoprecipitated using anti-Hsp70 antibody and the immunoprecipitates were incubated at 37° C. with alkaline phosphatase (AP; ThermoFisher) or at 30° C. with lambda phosphatase (LP; Santa Cruz Biotechnology) for 1 hr as described by the manufacturers. Phosphorylation status of untreated or phosphatase-treated Gli36 CM was verified by western blot analyses using anti-phosphotyrosine, anti-phosphoserine and anti-Hsp70 mAbs. For functional studies, 10× concentrated Gli36 CM was dephosphorylated without immunoprecipitation using AP or buffer. Dephosphorylated CM r buffer treated CM was passed through 10 kDa cut off filters to remove phosphatase buffers and used for testing THP-1 differentiation function.

### Subcutaneous Tumor Implantation

WT/Hsp70 knockdown LLC cells (1×10⁵) were injected subcutaneously into 6-8 weeks old C57BL/6J mice. Tumor growth was assessed daily by measuring tumor volume. When tumors reached 500 mm³, mice were euthanized and tumors were excised; half the tumor was frozen for histology and the reminder used for tumor macrophage analyses. For tumor growth analyses, 1×10⁵ LLC cells were injected subcutaneously into 6 week old TLR2^{-/-} mice or 1×10⁵ Rink1 cells were injected into 8 week old Axl^{-/-}, MerTK^{-/-}, and MerTK^{-/-}/Axl^{-/-} mice. Tumor growth and macrophage polarization were analyzed as described herein.

Tumor Dissociation into Single Cells and Flow Cytometry

Freshly excised tumors were cut into small pieces and minced using a scalpel. The minced tumor tissue was incubated with 100 units of collagenase Type 4 (Worthington Biochemicals, Lakewood, NJ) for 45 minutes at 37° C. in RPMI medium. The collagenase treated tumor tissue was passed through 40 μm cell strainer (ThermoFisher). The isolated tumor cells were washed twice with PBS then 5×10⁵ cells were incubated in 100 μl flow cytometry buffer (PBS+2% FBS) with mouse Fc block for 30 minutes on ice, followed by incubation with anti-mouse F4/80-PE (eBioscience) and anti-mouse CD206 APC (BioLegend) for 30 minutes on ice. Cells were washed and fixed with fixation buffer (eBioscience) for 30 minutes. Fixed cells were centrifuged and washed twice with PBS. Cells were permeabilized using permeabilization buffer (eBioscience) according to manufacturer's protocol and then incubated with anti-mouse iNOS antibody (eBioscience) in permeablization buffer for 45 minutes. Cells were washed twice with permeablization buffer and finally washed with flow cytometry buffer. Macrophages were then analyzed for M2 CD206+ cells and M1 iNOS+ cells by gating on F4/80 positive cells.

### Cell Sorting

Indicated cancer cell lines were stained with annexin V-FITC (Invitrogen, Carlsbad, CA) and PI, according to manufacturer's protocol. Briefly, 1×10⁶ cells were incubated with annexin V binding buffer (Invitrogen) together with PI for 30 mins at room temperature. Cells were washed with annexin V buffer, resuspended in this and cells with low annexin V signal and high annexin V signal were gated and sorted by flow cytometry.

### Immunofluorescence Staining

Cells were seeded on gelatin (0.01%)-coated coverslips. After a five hr incubation, cells were washed with PBS two times and fixed with 4% formaldehyde for 10 min at room temperature. After fixation, cells were washed two times with PBS and stained with primary anti-Hsp70 antibody at a 1:500 dilution (Abcam, Cambridge, United Kingdom) and anti-rabbit IgG (H+L), F(ab′)2 fragment AlexaFluor 555 conjugated at 1:1000 (Cell Signaling Technologies, Danvers, MA) according to the manufacturer's protocol. Cells were further stained with FITC-conjugated annexin V (ABP Biosciences, Beltsville, MD). After washing, the coverslips were finally mounted using the anti-fade reagent Fluoro-gel II with DAPI (Electron Microscopy Sciences, Hatfield, PA). Negative controls were stained with only anti-rabbit IgG or only mouse IgG-FITC (Santa Cruz Biotechnology). Samples were analyzed using a BX51 fluorescence microscope and the appropriate filters (Olympus, Tokyo, Japan). Images were captured with a cooled CCD camera using Spot Advanced software (Spot Imaging Solutions). Exposure times of fluorescence images were between 30 and 100 ms.

### Quantitative Co-Localization Analysis of Fluorescence Microscopy Images

Image J software was used to calculate the area of total cells and co-localization of PS and Hsp70. Ten randomly selected cells were used for calculations. Statistical analysis was performed using GraphPad Prism 6 Software and Microsoft Excel using unpaired student t-test.

### SDS-Page and Western Blotting Analyses

For SDS-PAGE, 50 μg of whole cell lysates in RIPA buffer (Sigma) were denatured in SDS-loading dye (Bio-Rad, Hercules, CA) then loaded onto 4-15% denaturing gradient gels (Bio-Rad). Proteins from the gel were transferred onto nitrocellulose membranes and blots were blocked with 5% milk in PBS-0.1% Tween-20®, followed by addition of protein specific antibodies and incubated overnight at 4° C. The blots were washed with PBS-Tween-20 three times and further incubated with HRP-coupled secondary antibodies. Following three washes with PBS-Tween-20®, the blots were developed with SuperSignal West Dura (ThermoFisher). For immunoprecipitates, the protein A+G agarose beads were boiled in SDS loading dye and loaded onto a gel and western blotting was performed.

### Statistical Analysis

GraphPad Prism 6 software was used for all statistical analyses. Data are presented as mean±SEM and were analyzed using a Student t-test (unpaired) for two group comparisons. Details of statistical analyses are provided in the figure legends. In vitro experiments were performed at least three times with consonant results. In vivo experiments were performed at least three times with consistent results. P values less than 0.05 were considered statistically significant.

### Example 2. Identification and Partial Characterization of Cancer-Secreted Py-Hsp70 that Induces Monocyte and Macrophage Differentiation and M2 Polarization Activity of Cancer Cell Conditioned Media (CM)

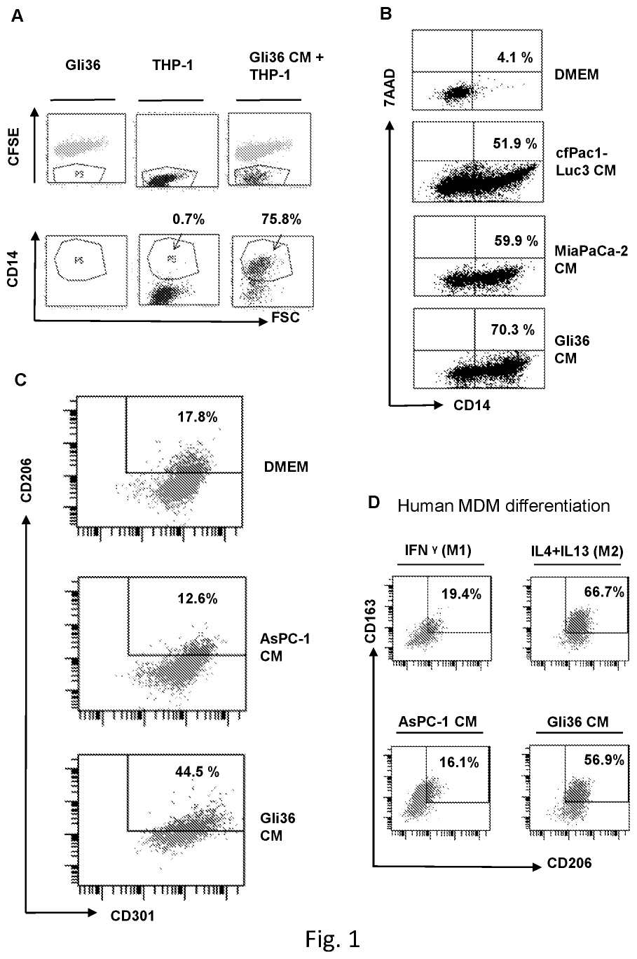

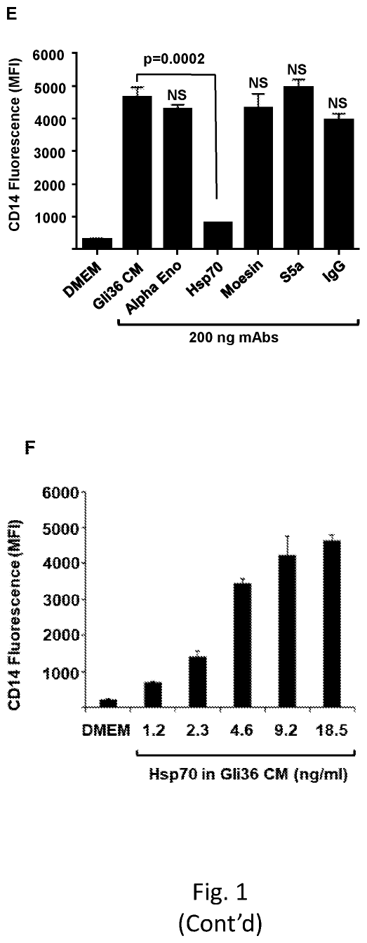

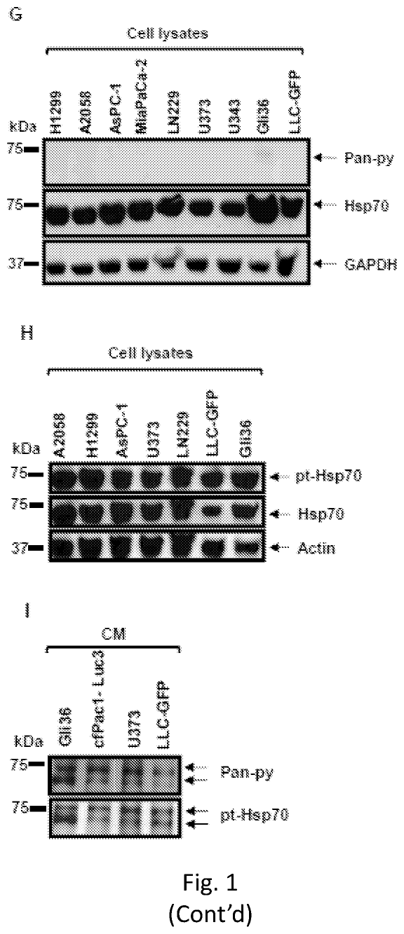

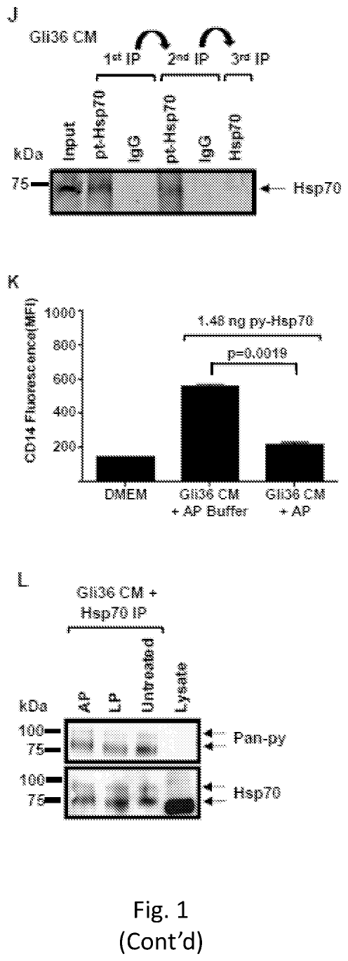

Many types of cancers are known to escape the host immune surveillance by inducing immunosuppressive M2 polarization of macrophages. Therefore, macrophage differentiation was analyzed in direct co-cultures of Gli36 cancer cells together with the widely accepted human monocyte cell line THP-1. To distinguish cancer cells from THP-1 cells, the former were labelled with carboxyfluorescein succinimidyl ester (CFSE) prior to co-culture. Gli36 cells induced robust differentiation of THP-1 cells as indicated by a pronounced elevation of the differentiation marker CD14 (FIG. 1A). Cancer-secreted cytokines help recruit macrophages to the tumor site and cancer-secreted factors like versican and protein S are known to promote macrophage M2 polarization. Therefore, to further assess whether cancer cells require direct contact with THP-1 cells, or trigger THP-1 differentiation via secreted factor(s), cancer conditioned medium (CM) from various cancer cell lines (Gli36, cfPac1-Luc3 and MiaPaCa-2) was added to THP-1 cultures. CM from cancer cell lines induced robust THP-1 differentiation, indicating soluble factors from cancer cells induce THP-1 differentiation (FIG. 1B).

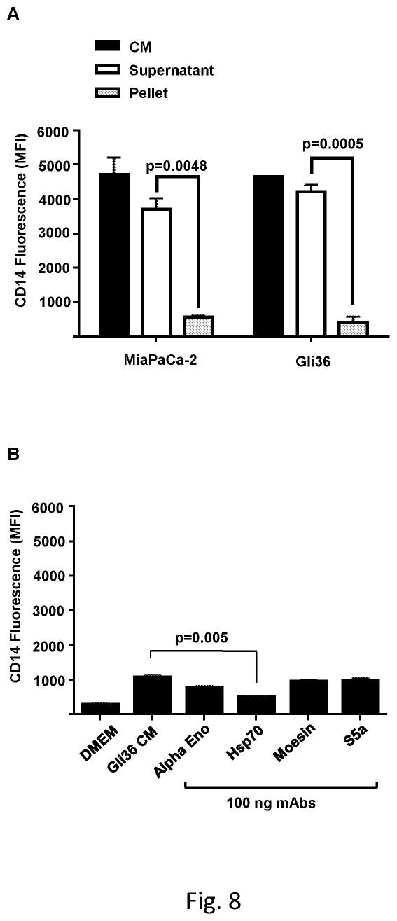

To assess whether THP-1 differentiation activity is present in the microparticle/exosomes fraction, ultracentrifugation of MiaPaCa-2 CM and Gli36 CM was carried out to separate these fractions. THP-1 cells were cultured with unfractionated CM, microparticle/exosome fraction or microparticle/exosome free fraction. The microparticle/exosome free fraction of CM contained major THP-1 differentiation activity (FIG. 8). Also tested was the ability of cancer cell CM to induce macrophage M2 polarization ability using the mouse macrophage cell line, J774, and primary human monocyte-derived macrophages (HMDM). Gli36 CM induced a strong increase in M2 polarization of mouse macrophage cell line J774 cells, as shown by increased expression of M2 markers CD206 and CD301 (FIG. 1C). While Gli36 CM was able to induce M2 polarization, CM from AsPC-1 cells was comparable to DMEM medium control, indicating that cancer cell types differ in their macrophage polarization capability.

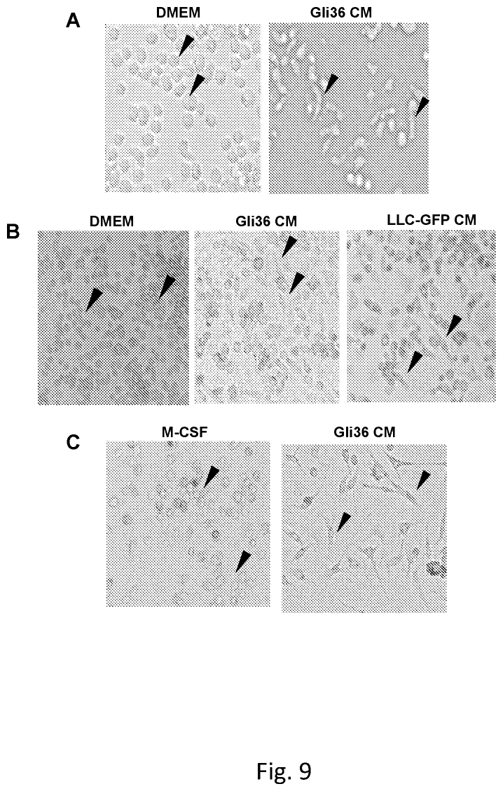

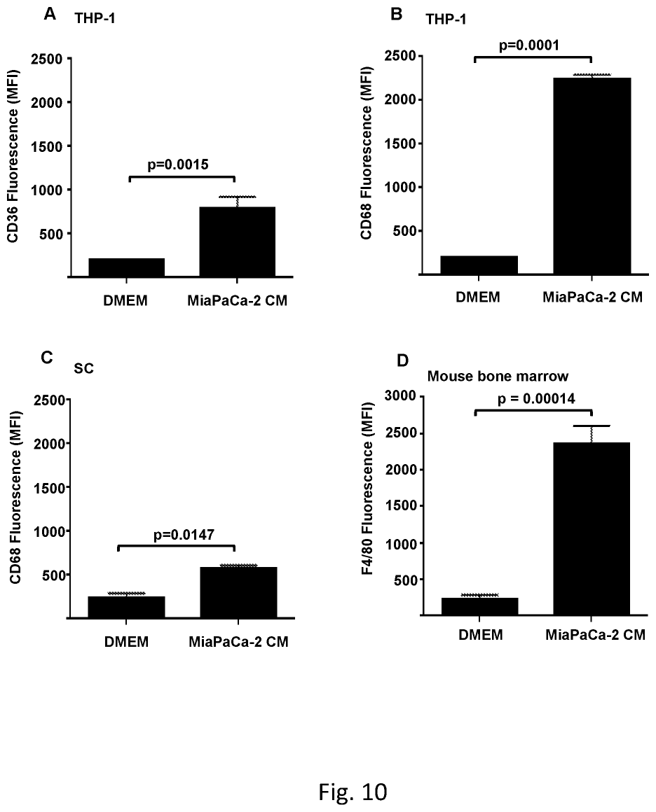

Next, the influence of Gli36 and AsPC-1 CM on M2 polarization of HMDMs was determined, since the effects seen in primary cells are more reliable than in cell lines. Gli36 CM induced an increase in M2 polarization, comparable to that seen by known M2 polarizing cytokines, IL-4 and IL-13. However, M2 polarization in response to AsPC-1 CM, was very low (FIG. 1D). Incubation of CM from cancer cell lines Gli36 and LLC-GFP with THP-1 monocytes, J774 macrophages and mouse bone marrow-derived macrophages (BMDMs) resulted in morphological changes with flattened and extended morphology, reminiscent of macrophages (FIG. 9). These morphological changes were not seen when these cells were incubated with normal cell culture medium. Consistent with these morphologic changes, CM from MiaPaCa-2 induced expression of macrophage differentiation markers, CD36 and CD68 on THP-1 cells, increased CD68 on SC macrophage cell line and pan macrophage marker, F4/80 on mouse bone marrow cells, indicative of macrophage differentiation (FIG. 10).

### Example 3. Identification of Cancer Secreted Py-Hsp70 as the Causal Factor Required for Macrophage Differentiation

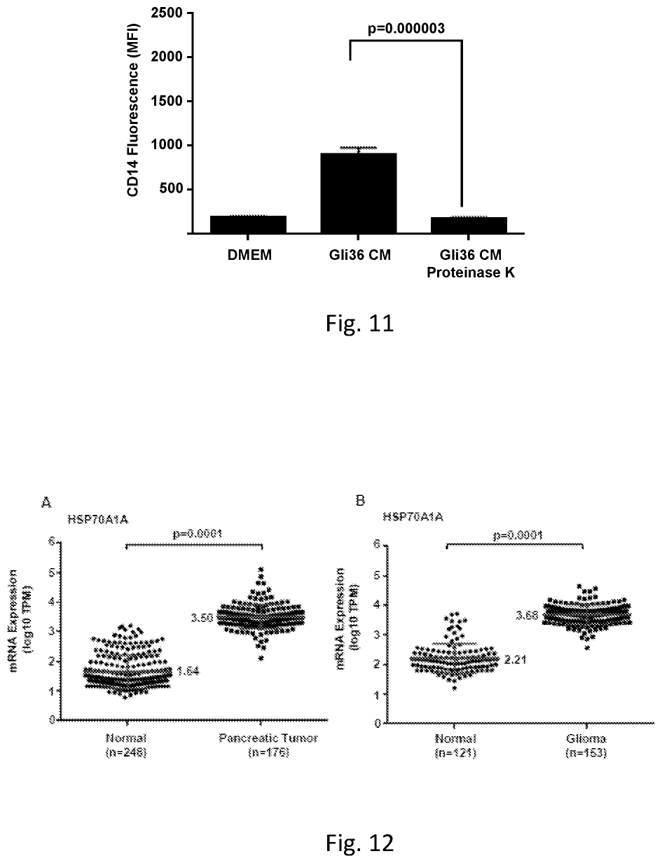

To determine if the secreted macrophage differentiation factor(s) is a protein, two interventions were performed using Gli36 CM. Heat inactivation of CM led to complete loss of THP-1 differentiation ability, suggesting the factor(s) is not an endotoxin. Next, to determine whether the factor(s) in the CM were proteinaceous, the CM was treated with Proteinase K. Such treatment led to complete loss of THP-1 differentiation activity of Gli36 CM (FIG. 11). Based on this evidence, LC-MS analyses of CMs from Gli36, MiaPaCa-2 and AsPC-1 were performed and, out of the many proteins identified, a small number of proteins were selected including Alpha Enolase, Hsp70, Moesin and SSA based on their known potential to regulate the immune system. The impact of monoclonal antibodies (mAbs) targeting these specific proteins was assessed on THP-1 differentiation, in response to Gli36 CM. Monoclonal antibodies against Hsp70 showed potent inhibition of Gli36 CM induced THP-1 differentiation indicating that cancer-secreted Hsp70 is required for THP-1 differentiation (FIG. 1E and FIG. 12B). Furthermore, increasing volumes of Gli36 CM were tested for THP-1 differentiation activity and found that Gli36 CM as low as 62.5 μl can induce THP-1 differentiation, with much higher activity obtained with 1 ml of CM (FIG. 1F).

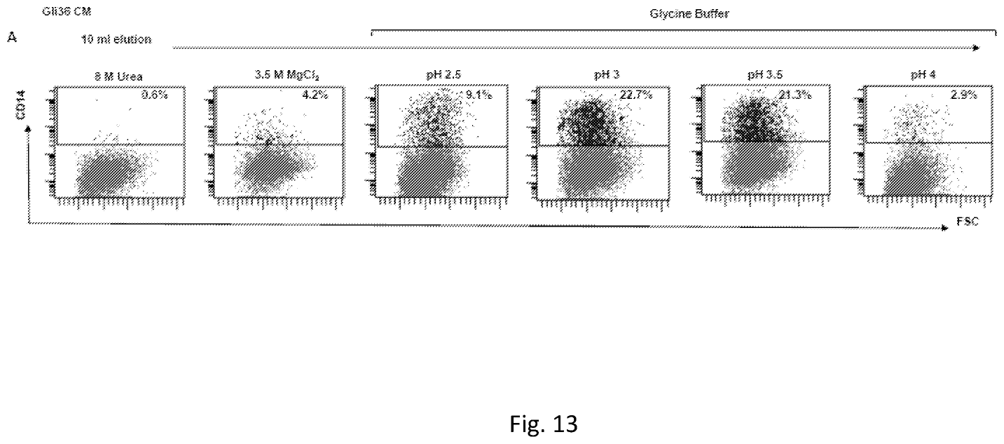

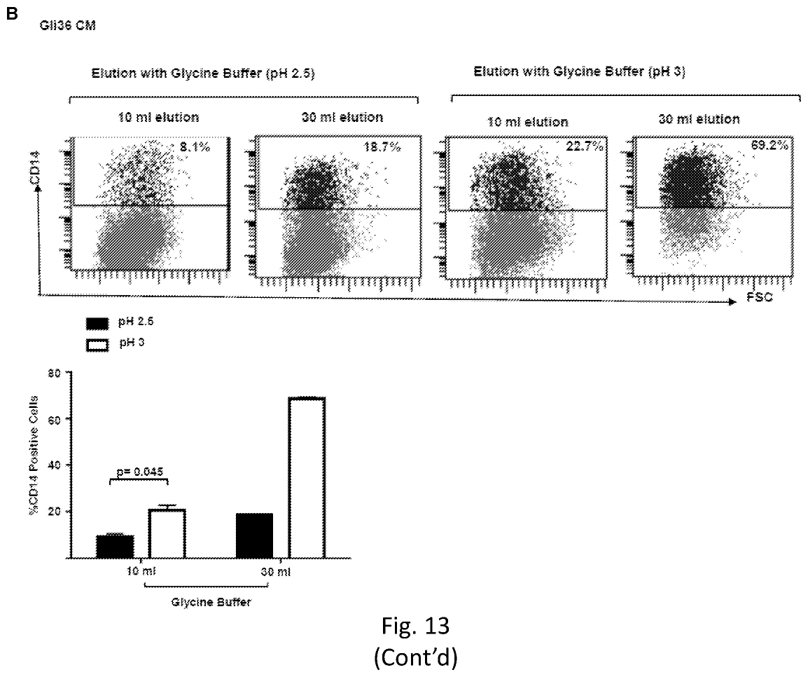

Although Hsp70 is a classical intracellular chaperone protein involved in protein folding, it is increasingly appreciated as multifunctional protein with a complex array of actions exerted both as intracellular and secreted protein. Human TCGA data set shows elevation of RNA expression of Hsp70 in Pancreatic and Glioma cancers (FIG. 13A-B). Further studies assessed the expression and also threonine and tyrosine phosphorylation status of Hsp70 in a variety of cancer cells. Total intracellular levels of Hsp70 are similar in different cancer cell lines (FIG. 1G, middle panel). Intracellular phosphothreonine Hsp70 (pt-Hsp70) levels, as measured by anti-phosphothreonine-Hsp70 specific mAb that recognizes phosphorylation on threonine 636 on Hsp70 are also similar in different cancer cell lines (FIG. 1H, upper panel). However, intracellular Hsp70 is not tyrosine phosphorylated as measured by a pan-phosphotyrosine specific mAb (Pan-py) but cancer-secreted Hsp70 is specifically tyrosine phosphorylated (py-Hsp70) (FIG. 1G upper panel and FIG. 1I upper panel). Both intracellular and secreted Hsp70 are threonine phosphorylated (pt-Hsp70) (FIG. 1H upper panel and FIG. 1I lower panel). These findings demonstrate that cancer-secreted Hsp70 is tyrosine phosphorylated (py-Hsp70) but intracellular Hsp70 is not, supporting a role for tyrosine phosphorylation in py-Hsp70 secretion and/or macrophage polarization function. To test whether the whole or only a fraction of secreted-Hsp70 is phosphorylated, serial immunoprecipitations of Gli36 CM were performed, using the phosphothreonine-Hsp70 specific mAb, to deplete pt-Hsp70 from Gli36 CM, followed by immunoprecipitation with an antibody that recognizes Hsp70 irrespective of its phosphorylation state (anti-Hsp70 mAb). These studies revealed that all of the secreted Hsp70 from Gli36 cells is phosphorylated, as evidenced by lack of Hsp70 in Gli36 CM after serial depletion with phosphothreonine-Hsp70 specific mAb (FIG. 1J). To test the functional relevance of py-Hsp70 phosphorylation in macrophage differentiation, cancer cell CM was subjected to dephosphorylation by alkaline phosphatase (AP) or Lambda phosphatase (LP). Both LP and AP partially dephosphorylated py-Hsp70 from CM (FIG. 1L) which led to a significant decrease in THP-1 differentiation compared to the untreated Gli36 CM, suggestive of the importance of py-Hsp70 phosphorylation in the macrophage differentiation (FIG. 1K). Since partially dephosphorylated Gli36 CM shows reduced THP-1 differentiation activity, and only cancer secreted Hsp70 is tyrosine phosphorylated, it suggests tyrosine phosphorylated sites on py-Hsp70 are involved in THP-1 differentiation activity.

Additional studies examined whether semi-purified py-Hsp70 from Gli36 CM maintained macrophage differentiation activity. Following immunoprecipitation of Hsp70 from Gli36 CM by using anti-Hsp70 mAb, different methods of elution of py-Hsp70 from the immunoprecipitates were performed, using 8 M urea, 3.5 M MgCl₂ or low pH glycine buffer (with pH 2.5, pH 3, pH 3.5, and pH 4) (FIG. 13A). Hsp70 elution using glycine buffer with acidic pH (pH 2.5, pH 3, and pH 3.5) showed intact activity with highest activity obtained from glycine buffer pH 3 eluates after neutralization of pH (FIG. 13A, B). Elution accomplished with pH 4, MgCl₂ or 8 M urea were inactive even after renaturation by dialysis (FIG. S6A). Eluates of Hsp70 obtained from Hsp70 immunoprecipitates of 10 ml, and 30 ml of Gli36 CM showed increasing activity of THP-1 differentiation (FIG. 13B). Together these data indicate that partially purified Hsp70 from Gli36 CM retains THP-1 differentiation activity.

### Example 4. Hsp70 Knockdown in Cancer Cells Impairs Cancer Cell-Induced Macrophage Differentiation, Decreases Tumor Growth in Mice, and Alters Intra-Tumor Macrophage Polarization

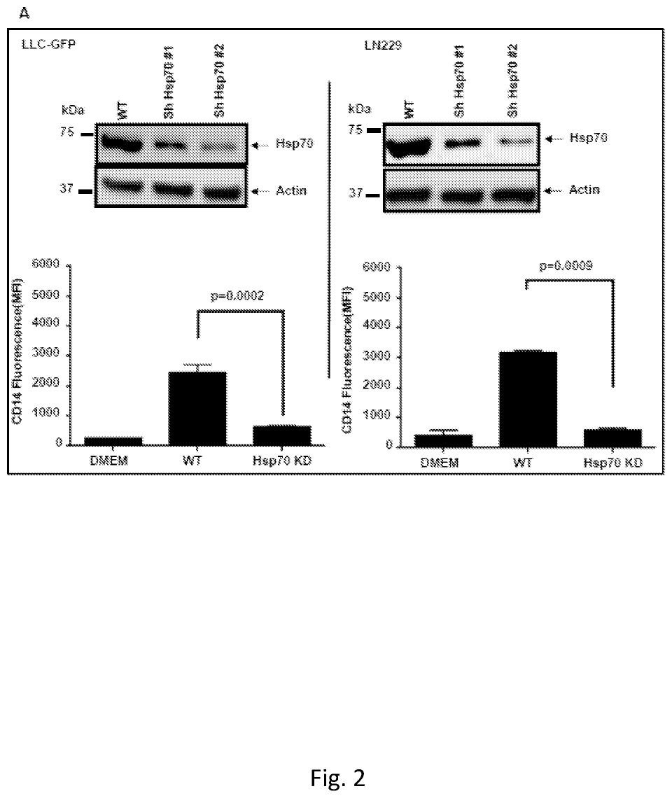

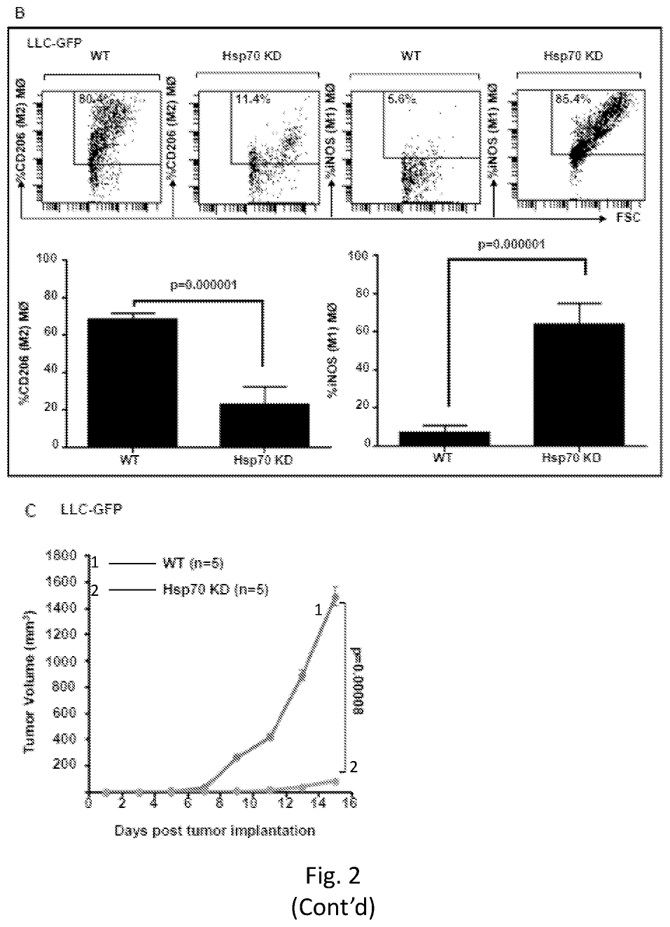

Having established that py-Hsp70 is a critical factor for THP-1 differentiation, and that phosphorylation is likely required for py-Hsp70 function, further studies explored the influence on macrophage differentiation, tumor growth and intra-tumor macrophage polarization by knocking down total Hsp70 in two different mouse cancer cell lines (LLC-GFP and LN229) to study these parameters in mouse sub-cutaneous tumors. Hsp70 knockdown was carried out in LLC-GFP and LN229 cells by lentiviral-mediated expression of either control shRNAs or shRNAs targeting Hsp70 (FIG. 2A). In vitro, Hsp70 Knockdown LLC-GFP and LN229 cells are viable and grew comparable to WT cells, most likely because the knockdown was partial and knockdown cells still express Hsp70, albeit at much lower level than WT cells (FIG. 2A). Knockdown of Hsp70 in LLC-GFP and LN229 cells led to a marked decrease in macrophage differentiation activity of the CM obtained from LLC-GFP and LN229 cells compared to CM obtained from control shRNA expressing cells, as evident by strong reduction in CD14 expression on THP-1 cells (FIG. 2A). Of note, when these cells were implanted subcutaneously into mice, tumor growth from LLC-GFP cells with Hsp70 knockdown was severely impaired compared to LLC-GFP cells with control shRNAs (FIG. 2C). Most importantly, when intra-tumor macrophages were assessed for their polarization status, tumors from LLC-GFP cells with control shRNAs (WT, contained predominantly pro-tumorigenic M2 polarized macrophages, while tumors from Hsp70 knockdown cells are very small and contained M1 polarized macrophages, while WT tumors had low M1 macrophages compared to Hsp70 KD tumors (FIG. 2B). These results suggest the importance of py-Hsp70 in the regulation of tumor macrophage polarization and subsequent tumor growth.

### Example 5. Cancer-Secreted Py-Hsp70 Induces Macrophages to Secrete Chemokines/Cytokines Implicated in Macrophage Recruitment and Polarization

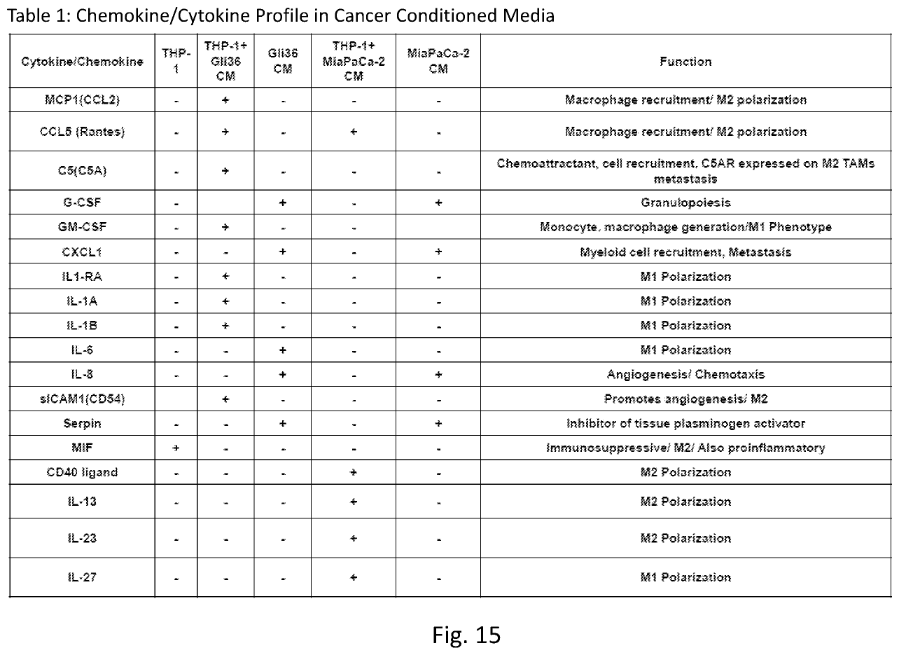

The data strongly suggest an important role for py-Hsp70 in macrophage M2 polarization. To better understand the pathways involved in macrophage M2 polarization, additional studies measured the chemokine/cytokine profile induced by cancer cell CM using Gli36 and MiaPaCa-2 CM. It was hypothesized that the specific fingerprint might suggest which chemokine pathways are stimulated and indicate the specific cell surface receptors involved. Interestingly, it was found that THP-1 cells treated with Gli36 CM or MiaPaCa-2 CM secreted MCP-1, CCL5, sICAM-1, which are involved in macrophage recruitment and M2 polarization and complement factor C5A involved in M2 polarization (FIG. 15). In addition, secretion of factors involved in M1 polarization such as IL-1, IL-6, IL-8 and IL-27 was observed. These data suggest that py-Hsp70 may directly stimulate a number of key signaling pathways involved in macrophage recruitment and polarization.

### Example 6. Py-Hsp70 Acts Through Macrophage TLR2 and MerTK Receptors to Induce Macrophage Differentiation and Polarization

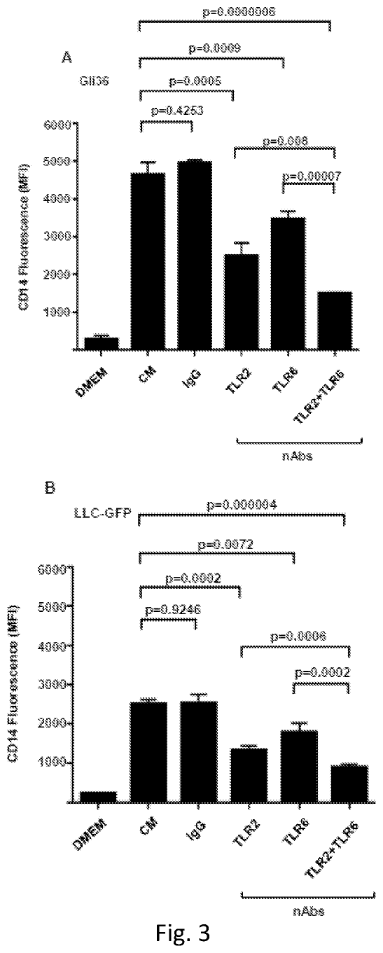

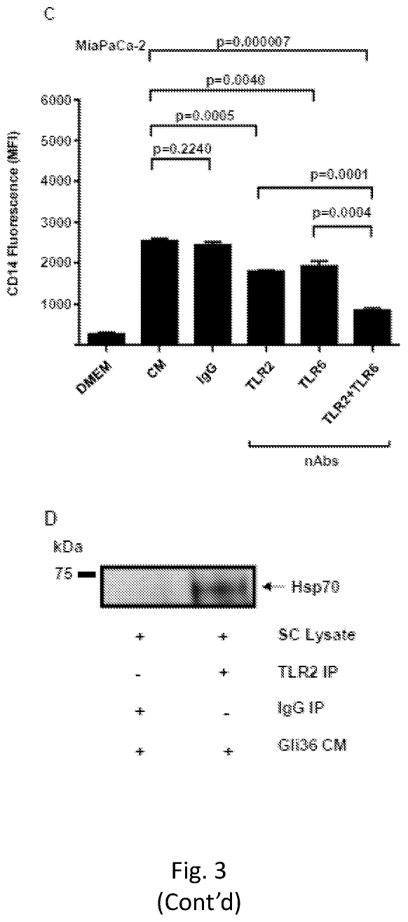

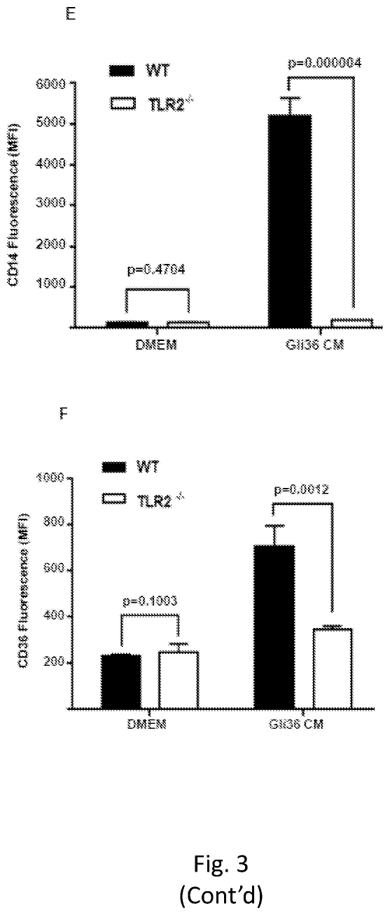

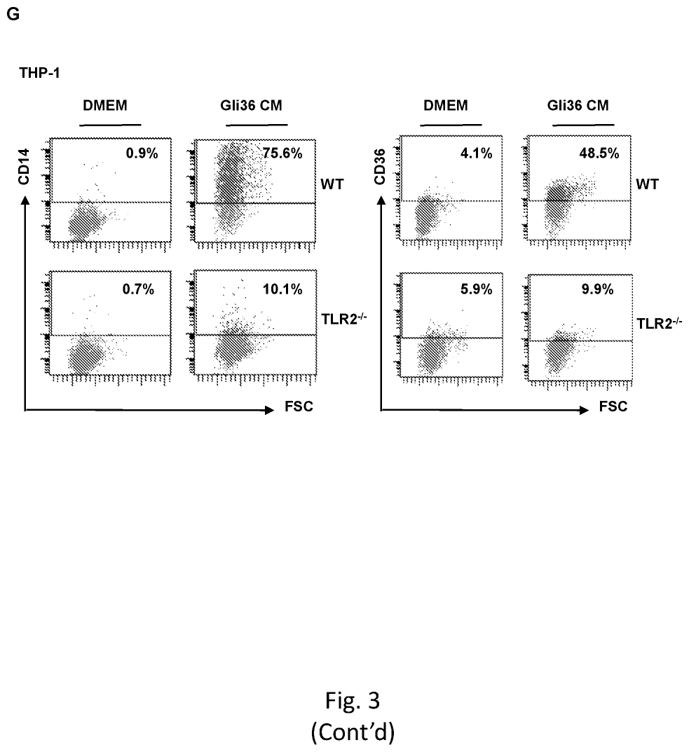

Hsp70 is known to bind TLRs. Therefore, to assess the role of TLRs in cancer-CM induced macrophage differentiation, a panel of TLR neutralizing antibodies (nAbs) was added to cultures of THP-1 cells together with Gli36 CM. Inhibition of THP-1 differentiation was observed specifically by TLR2 and TLR6 nAbs (data not shown). Neutralizing antibodies against TLR2 and TLR6 together strongly impaired THP-1 differentiation induced by CM from cancer cell lines Gli36, LLC-GFP, and MiaPaCa-2 (FIG. 3A-C). This suggests that TLR2 and TLR6 are required for the induction of THP-1 differentiation by the CM (FIG. 3A-C). Since a TLR2 requirement for cancer-CM induced macrophage differentiation was identified, studies examined whether py-Hsp70 directly binds to TLR2. To test this, TLR2 was immunoprecipitated from macrophages and incubated with Gli36 CM and detected py-Hsp70 in TLR2 immunoprecipitates, suggesting the interaction of py-Hsp70 with TLR2 (FIG. 3D). To further confirm the requirement of TLR2 in macrophage differentiation induced by cancer CM, TLR2 null THP-1 cells were used in cultures with Gli36 CM. As expected, Gli36 CM failed to induce differentiation of TLR2 null THP-1 cells, as evidenced by inhibition of CD14 and CD36 expression while WT THP-1 cells had a strong increase in both CD14 and CD36 markers after addition of CM (FIG. 3E-G). These data suggest that py-Hsp70 induces macrophage M2 polarization through direct activation of TLR2/6 receptors.

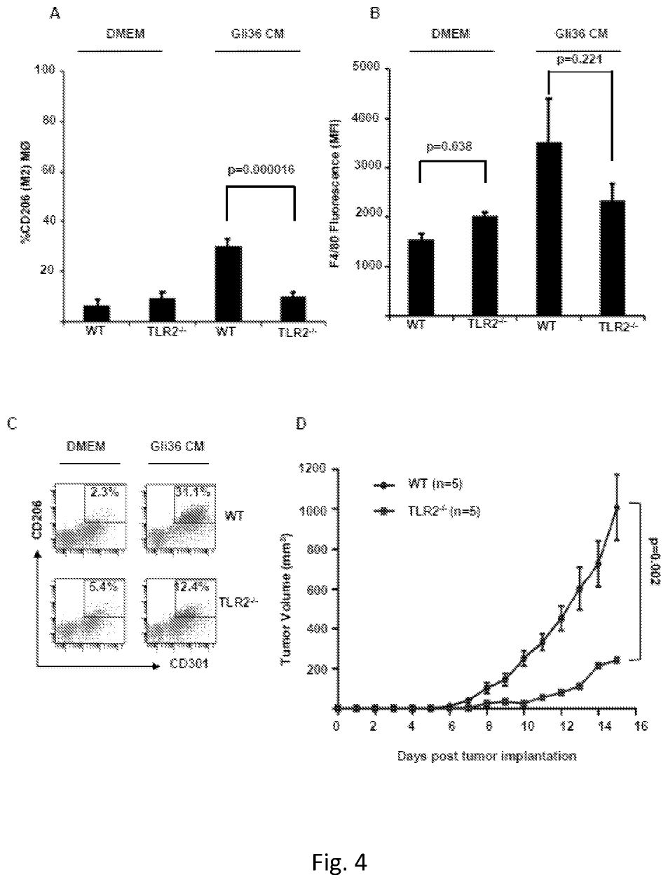

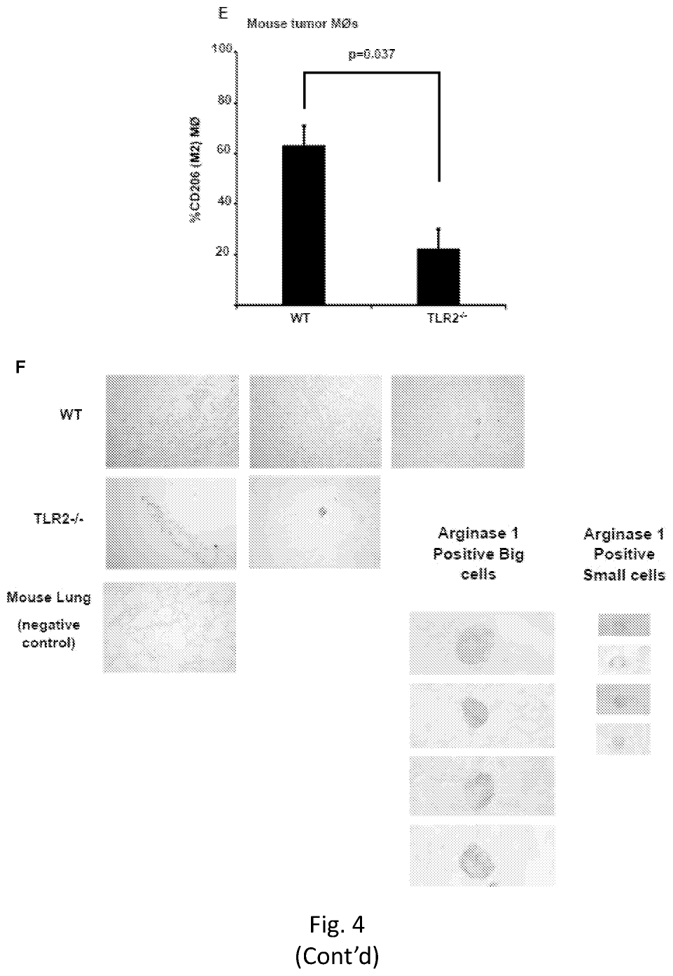

To further investigate the TLR2 requirement in vivo for Gli36 CM-induced macrophage polarization, Gli36 CM was injected into peritonea of WT or TLR2 null mice. 24 hrs later the peritoneal macrophages were isolated and analyzed by flow cytometry. As shown in FIG. 4A, Gli36 CM induction of peritoneal macrophage M2 polarization was significantly reduced in TLR2 null mice compared to WT mice, but there was no significant reduction of cells stained by the pan macrophage marker, F4/80 (FIG. 4B), suggesting a specific effect on M2 polarization (FIG. 4A-C). Since there was an interaction between py-Hsp70 and TLR2, following studies determined whether TLR2 is required for tumor growth and tumor macrophage polarization in mice. To achieve this, mouse lung cancer cell line LLC-GFP cells were subcutaneously implanted in WT mice and TLR2 null mice and growth and macrophage polarization were monitored. As shown in FIG. 4D, tumor growth in TLR2 null mice is slower compared to WT mice. Further, the percentage of intra-tumor M2 polarized macrophages was substantially reduced in TLR2 null mice (FIG. 4E). These data indicate the importance TLR2-mediated macrophage polarization in tumor growth control. Immunohistochemistry of tumor sections revealed increased presence of Arginase1 positive macrophages in tumors of WT mice compared to TLR2^{-/-} mice (FIG. 4F).

### Example 7. Py-Hsp70 Induces Upregulation of MerTK Through TLR2 and MerTK Assembly on Macrophages

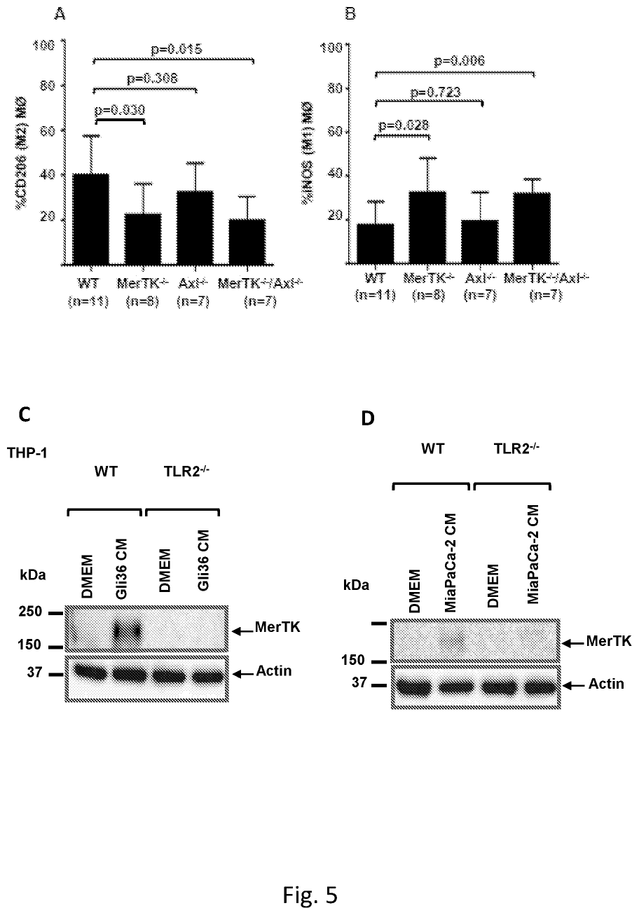

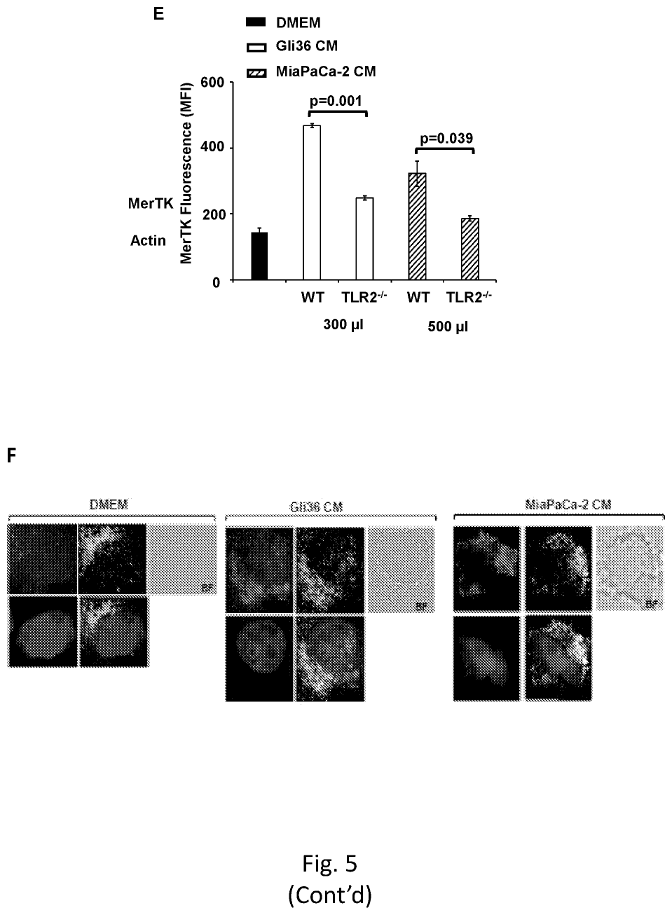

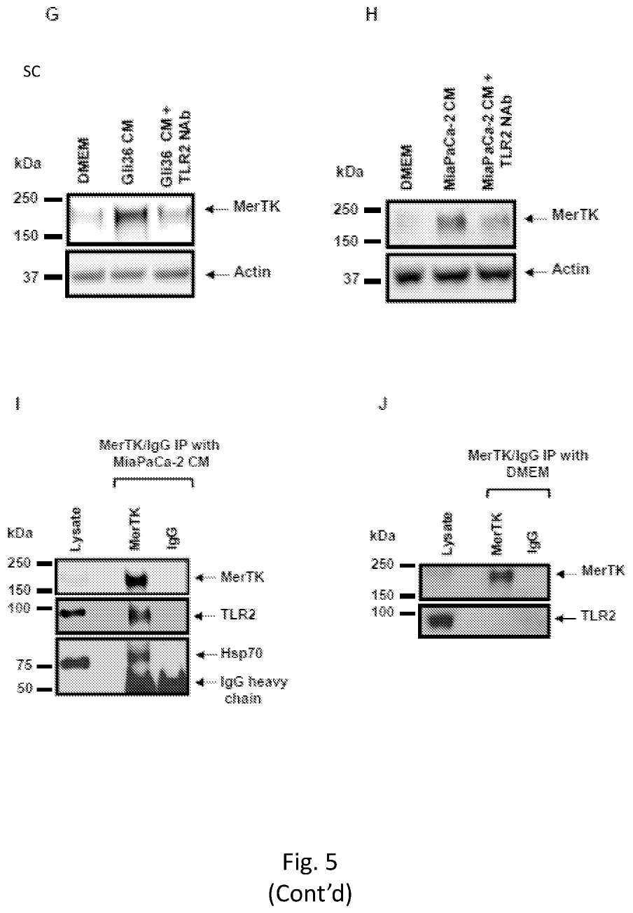

Although our findings indicate a requirement of TLR2 for cancer CM-induced macrophage M2 polarization, macrophage Tyro3, Axl and MerTK on macrophages are well known for regulation of macrophage polarity. Additional studies examined the requirement of Tyro3, Axl and MerTK in Gli36 CM-induced macrophage M2 polarization by using macrophages obtained from peritonea of MerTK null, Axl RTK null and WT mice. As shown in FIG. 5A, B, Gli36 CM-induced M2 polarization of peritoneal macrophages in MerTK null mice was significantly reduced compared to WT and Axl null mice, indicating specific requirement of MerTK receptors. Interestingly, the reduction in M2 polarized macrophages in MerTK null mice is accompanied by a concomitant increase in M1 macrophages (FIG. 5B). Such a phenotype shift makes MerTK a vulnerable therapeutic target. Taken together, these results support a requirement of both TLR2 and MerTK receptors in Gli36 CM-induced macrophage M2 polarization, since a lack of either TLR2 or MerTK resulted in a significant reduction in macrophage M2 polarization.

Although the TLR system has not been previously directly linked to Tyro3, Axl, and MerTK receptors, these results indicated that both TLR2 and MerTK are required for cancer CM-induced macrophage M2 polarization, supporting a potential collaboration of TLR2 and MerTK receptors. Therefore, the effect of Gli36 CM on MerTK at the expression level was tested. Robust induction of the MerTK receptor in THP-1 cells was detected in response to Gli36 and MiaPaCa-2 CM, in WT RTHP-1 cells but not in TLR^{-/-} THP-1 cells, indicating the critical role of TLR2 in regulating the expression of MerTK receptors (FIG. 5C-E). Also observed was upregulation of MerTK in SC macrophage cell line in response to Gli36 or MiaPaCa-2 CM that is inhibited by TLR2 neutralizing antibodies (FIG. 5G, H). Further studies examined if TLR2 and MerTK co-localize on the membranes of THP-1 cells in response to cancer CM by immunofluorescence staining. Clearly, Gli36 CM or MiaPaCa-2 CM enabled profound co-localization of TLR2 and MerTK receptors compared to DMEM medium treated cells (FIG. 5F). Further studies analyzed whether the cancer-secreted py-Hsp70 associates with TLR2 and MerTK, through co-immunoprecipitation from Gli36 CM-stimulated macrophage using a MerTK monoclonal antibody. Results showed that both py-Hsp70 and TLR2 are present in the MerTK immunoprecipitates, indicating py-Hsp70 assembles with TLR2 and MerTK (FIG. 5I). Such assembly of MerTK and TLR2 was absent when DMEM was substituted for Gli36 CM (FIG. 5J). Together, these data indicate that py-Hsp70 binds to TLR2 and associates with MerTK on macrophages and triggers upregulation of MerTK, and facilitating amplification of immunosuppressive signal from macrophages, through upregulated MerTK.

### Example 8. High Surface PS on Cancer Cells Promotes Macrophage Differentiation and M2 Polarization

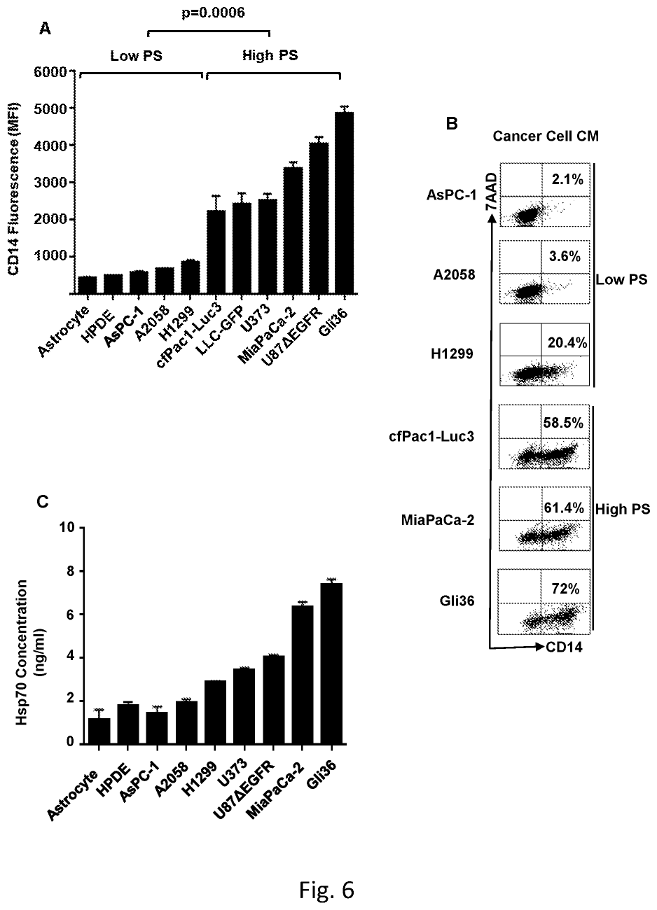

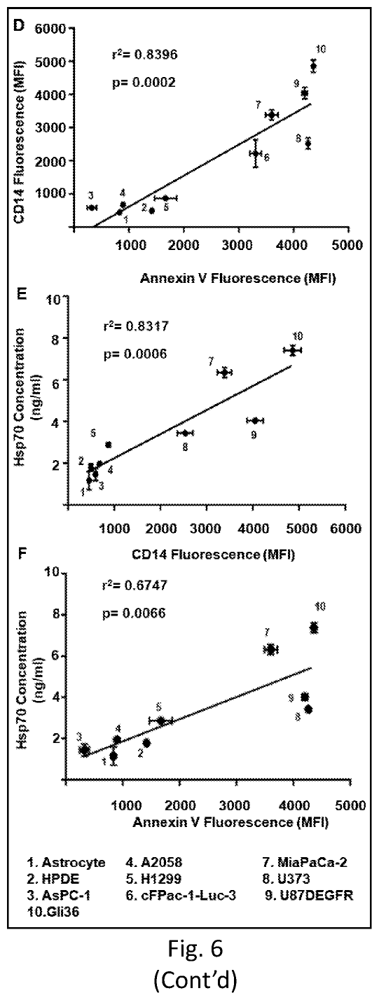

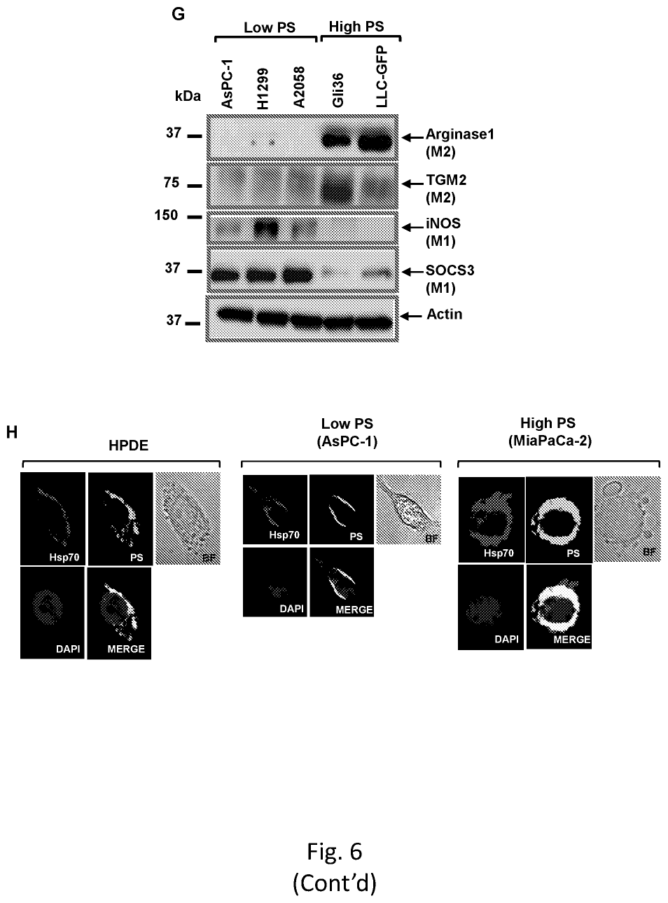

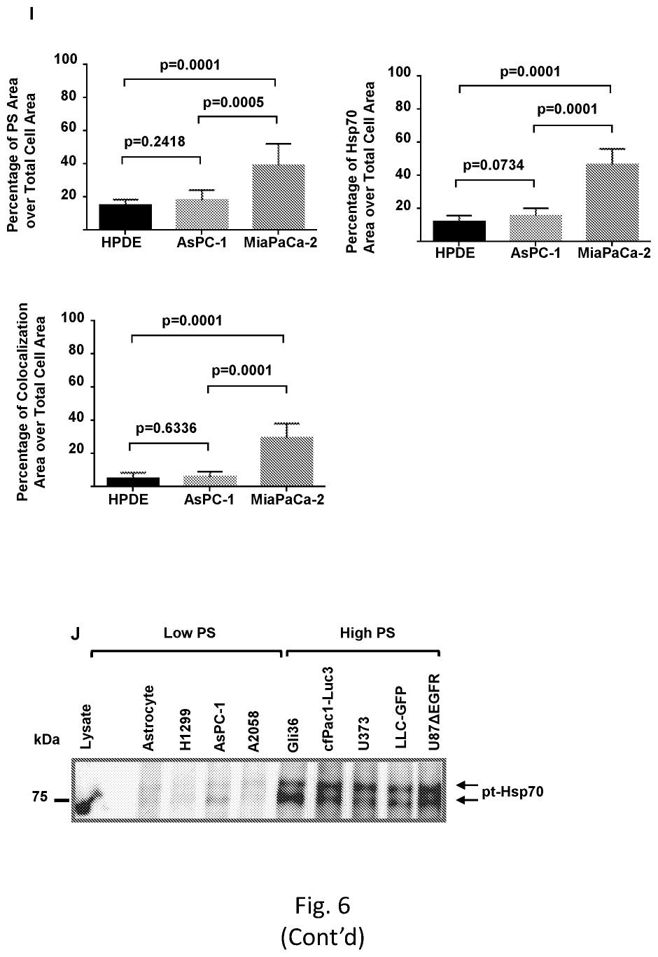

The findings that py-Hsp70 released from cancer cells promotes an immunosuppressive tumor microenvironment raised the question of what regulates the secretion in apparent greater abundance in cancer cells. The surface lipid phosphatidylserine (PS) could be a potential candidate in this process as PS is known to bind Hsp70, PS is over-exposed on the surface of cancer cells and linked to increased cancer malignancy. Further, Hsp70 is known to be secreted in a membrane raft-associated manner, where PS is an important component. Macrophages express PS receptors and PS on target cells recognized by macrophages is known to regulate macrophage functions. Given the strong evidence that tumor-derived py-Hsp70 is potential mediator of TME immune suppression, further studies explored the mechanism for py-Hsp70 secretion. Hsp70 is known to be secreted by a non-classical secretory pathway. Therefore, the importance of cancer cell surface PS in secretion of py-Hsp70 and macrophage differentiation was examined by evaluating CM obtained from low surface PS and high surface PS cancer cell lines, on THP-1 differentiation. As shown in FIGS. 6A, B, and D, CM from cancer cell lines with high surface PS induced robust THP-1 differentiation as indicated by a strong increases in the differentiation marker CD14, compared to low surface PS cancer cell lines and primary human astrocytes and primary human HPDE cells. In addition, J774 macrophage cells treated with CM from high surface PS cancer cell line Gli36 and LLC-GFP cells showed marked increases in the M2 polarization markers, Arginase1 and TGM2, with corresponding decrease in the M1 markers NOS2, and SOCS3, compared to low surface PS cell lines AsPC-1 and H1299 (FIG. 6G).

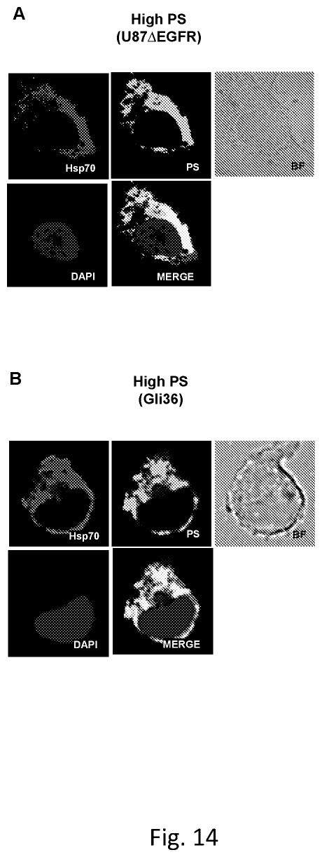

Since py-Hsp70 was observed as the critical factor in CM required for macrophage differentiation, ELISA and western blot analyses were used to determine the presence of py-Hsp70 in CM from low and high surface PS cancer cell lines and found the presence of higher levels of py-Hsp70 in the CM of high surface PS cancer cells (FIG. 6C, J). Interestingly, a strong direct correlation was observed between cancer cell surface PS, Hsp70 secretion, and induction of THP-1 differentiation (FIG. 6D-F, J). Since Hsp70 has been shown to interact with cell surface PS and high surface PS cancer cells secrete more py-Hsp70, additional studies assessed whether py-Hsp70 and membrane PS co-localize on the cell surface. The extent of co-localization of PS and py-Hsp70 was compared in immortal normal human pancreatic duct epithelial cells (HPDE), low surface PS AsPC-1 cells, high surface PS MiaPaCa-2, Gli36 cells and U87ΔEGFR cells (FIG. 6H, I and FIG. 14). Specific regions were observed showing co-localization of Hsp70 with membrane PS, with more co-localization in high surface PS cancer cell lines with higher area of co-localization, compared to normal HPDE cells and low surface PS AsPC-1 cancer cells. These results show clear correlations between the amount of cancer surface PS, co-localization of PS and Hsp70, the amount of secreted py-Hsp70, and macrophage differentiation.

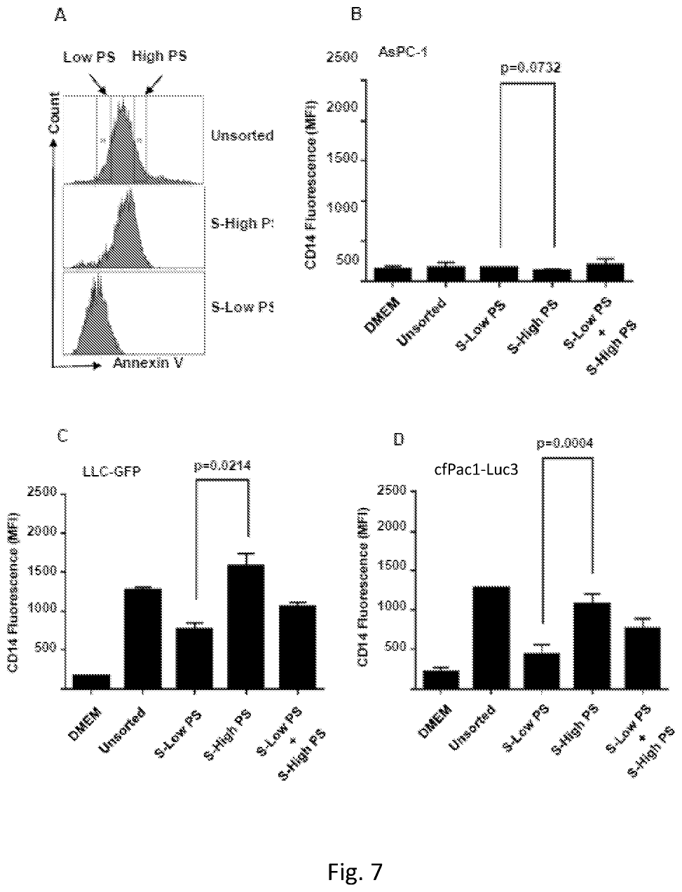

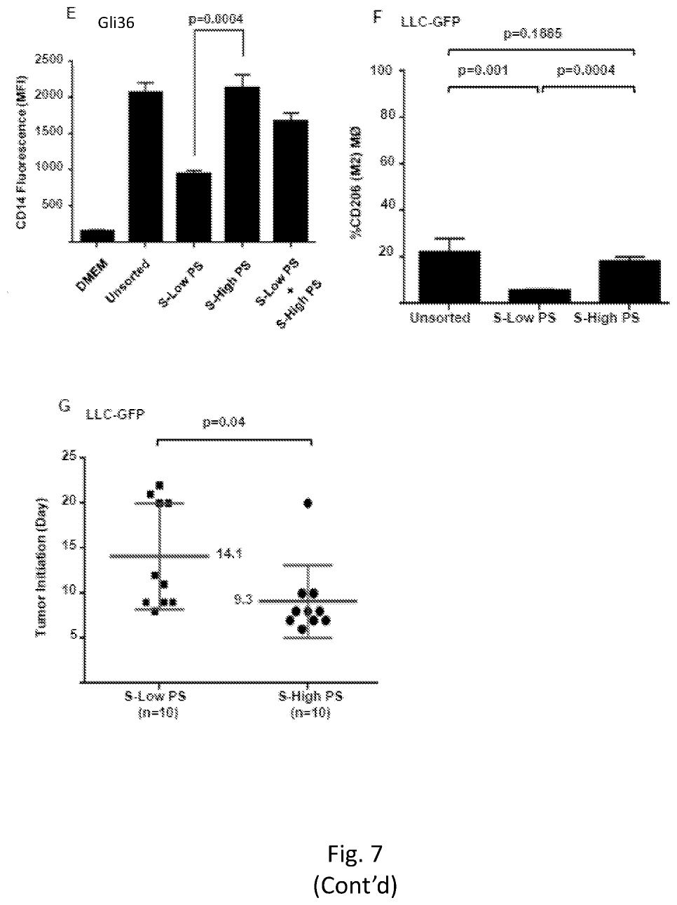

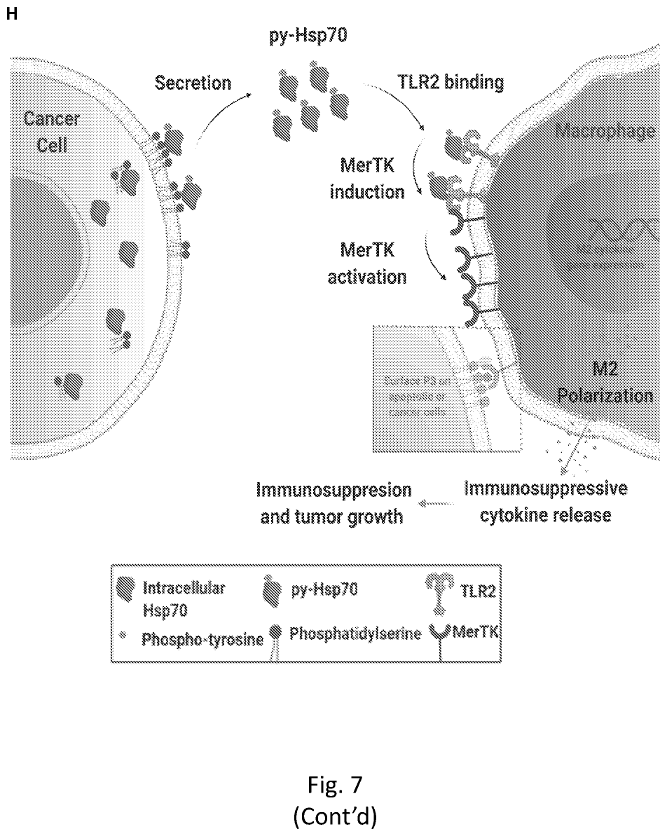

These results indicated that high surface PS cancer cells secrete more py-Hsp70 (FIG. 6A-F). However, it is not clear whether all the cells of individual cancer cell lines secreted macrophage differentiation activity or if the activity is due to a particular subpopulation of cells. Since recent studies reveal that tumor cells are highly heterogeneous with distinct cellular subsets and distinct functions, it was hypothesized that subpopulations differ in surface PS and secretion of macrophage py-Hsp70. To test this possibility, high surface PS (Gli36, LLC-GFP, cfPac1-Luc3) and low surface PS (AsPC-1) cell lines were sorted into higher surface PS and lower surface PS subsets by flow cytometry after annexin V staining (FIG. 7A). Unsorted, sorted higher surface PS, sorted lower surface PS, and a mixture of sorted higher and lower surface PS cells from the individual cell lines were then tested for their ability to induce THP-1 differentiation (FIG. 7B-E). Clearly, the sorted higher surface PS cells from high PS cell lines (Gli36, LLC-GFP, cfPac1-Luc3) exhibit higher THP-1 differentiation capacity compared to sorted lower surface PS cells from the same cell lines (FIG. 7B-E). However, the sorted higher surface PS cells from low PS cell line AsPC-1 failed in induction of THP-1 differentiation, possibly due to the overall lower surface PS (FIG. 7B). Together, these findings reveal the presence of novel PS-based immunoregulatory subpopulations in cancer cell lines.

Next, studies tested whether these populations possess differential macrophage polarization and tumor initiation ability in vivo. It was observed that sorted higher surface PS LLC-GFP cells induced stronger M2 polarization when injected into peritonea of C57Bl/6J mice compared to sorted lower surface PS LLC-GFP cells (FIG. 7F). Moreover, it was found that sorted higher surface PS LLC-GFP cells formed tumors earlier than lower surface PS LLC-GFP cells, when injected subcutaneously into immunocompetent C57BL/6J mice (FIG. 7G). These results indicate that high surface PS on cancer cells favors surface PS-associated secretion of py-Hsp70, which in turn acts on TLR2 receptors on macrophages, leading to robust upregulation of MerTK receptors on macrophages and formation of immunosuppressive molecular assembly of py-Hsp70-TLR2 and MerTK favoring macrophage M2 polarization and promoting tumor initiation through elevated secretion of py-Hsp70 (FIG. 711).

Patents, applications, and publications mentioned in the specification are indicative of the levels of those skilled in the art to which the invention pertains. These patents and publications are incorporated herein by reference to the same extent as if each individual application or publication was specifically and individually incorporated herein by reference.

The foregoing description is illustrative of particular embodiments of the invention, but is not meant to be a limitation upon the practice thereof. The following claims, including all equivalents thereof, are intended to define the scope of the invention.

What is claimed is:

1. A method of non-invasively diagnosing and treating a cancer characterized by high surface phosphatidylserine (PS) expression in a subject in need thereof, the method comprising:
obtaining a liquid biological sample from the subject;
detecting a presence of cancer-secreted soluble phosphorylated Heat shock protein-70 (Hsp70) in the liquid biological sample;
diagnosing the subject with the cancer characterized by high surface PS expression when cancer-secreted soluble phosphorylated Hsp70 is present in the liquid biological sample; and
administering an anti-cancer therapy targeted to high surface PS expressing-cancers to the diagnosed subject.

2. The method according to claim 1, wherein the liquid biological sample is selected from the group consisting of blood, serum, plasma, urine, breastmilk, saliva, tears, and sweat.

3. The method according to claim 1, wherein the secreted phosphorylated Hsp70 is tyrosine-phosphorylated Hsp70 (py-Hsp70).

4. The method according to claim 1, wherein the cancer characterized by high surface PS expression is selected from the group consisting of pancreatic cancer, glioma, melanoma, lung cancer, colorectal cancer, and pediatric cancers.

5. The method according to claim 1, wherein the anti-cancer therapy targeted to high surface PS expressing-cancers is selected from the group consisting of bavituximab, Saposin C-dioleoylphosphatidylserine (SapC-DOPS), phosphatidylcholine-stearylamine (PC-SA), DPA-CY3 1-palmitoyl-2-oleoyl-sn-glycero-3-phosphocholine (DPA-CY3/POPC), Chalepin, human annexin-V, PGN634, mch1N11, PPS1, PPS1D1, PSBP-6, hexapeptide E3, TSR-022, MBG543, BMS-986258, LY3321367, Sitravatinib, R428, TP0903, BMS-777607, NPS1034, MRX-2843, UNC2025, UNC3133, ONO-7475, Tyro3 inhibitors, 5F9, and combinations thereof.

6. The method according to claim 1, wherein the method further comprises detecting the presence of one or more additional biomarkers in the liquid biological sample.

7. The method according to claim 6, wherein the one or more additional biomarkers is selected from the group consisting of Heat shock protein-90 (Hsp90), moesin, S5a, polyubiquitin-B, ubiquitin-60S ribosomal protein L40, beta-hexosaminidase, neuronal pentraxin-1, variant surface antigen D, fibulin-1, nidogen-1, alpha enolase, Cyr61, PAI1, EF1 alpha, IGFBP3, versican, and sulfydryl oxidase.

8. The method according to claim 1, further comprising administering to the subject one or more additional anti-cancer therapeutics selected from the group consisting of an Hsp70 inhibitor, a Toll-like receptor 2 (TLR2) inhibitor, and a Mer tyrosine kinase (MerTK) inhibitor.

9. The method according to claim 8, wherein the Hsp70 inhibitor is selected from the group consisting of apoptozole, JG-13, JG-98, MAL3-101, MKT-077, spergualin, YM-01, YM-08, methylene blue, and combinations thereof.

10. The method according to claim 8, wherein the TLR2 inhibitor is selected from the group consisting of C29, ortho-vanillin, AT5, CUCPT-22, MMG-11, T2.5, OPN-305, and combinations thereof.

11. The method according to claim 8, wherein the MerTK inhibitor is selected from the group consisting of UNC569, UNC1062, UNC4203, UNC2025, UNC2250, MRX-2843, ONO-7475, RXDX-106, 549076, merestinib, and combinations thereof.

12. The method according to claim 8, wherein the Hsp70 inhibitor inactivates py-Hsp70.

13. The method according to claim 12, wherein the Hsp70 inhibitor is Saposin C dioleoylphosphatidylglycerol (SapC-DOPG).

14. The method according to claim 1, wherein administering comprising administering to the tumor microenvironment.

15. The method according to claim 1, further comprising administering to the subject one or more additional anti-cancer agents selected from the group consisting of chemotherapeutic agents, radiotherapeutic agents, cytokines, anti-angiogenic agents, apoptosis-inducing agents, and anti-cancer immunotoxins.

16. A method for monitoring therapeutic efficacy of a treatment in a subject with a cancer characterized by high surface phosphatidylserine (PS) expression, the method comprising:
treating the subject with an anti-cancer therapy targeted to high surface PS expressing-cancers;
measuring a level of cancer-secreted soluble tyrosine-phosphorylated Hsp70 (py-Hsp70) in a liquid biological sample obtained from the subject after administering the anti-cancer therapy; and
altering dosage of the anti-cancer therapy, frequency of dosing the anti-cancer therapy, or course of therapy administered to the subject based on the level of cancer-secreted soluble py-Hsp70 measured.

17. The method according to claim 16, wherein the anti-cancer therapy targeted to high surface PS expressing-cancers is selected from the group consisting of bavituximab, Saposin C-dioleoylphosphatidylserine (SapC-DOPS), phosphatidylcholine-stearylamine (PC-SA), DPA-CY3 1-palmitoyl-2-oleoyl-sn-glycero-3-phosphocholine (DPA-CY3/POPC), Chalepin, human annexin-V, PGN634, mch1N11, PPS1, PPS1D1, PSBP-6, hexapeptide E3, TSR-022, MBG543, BMS-986258, LY3321367, Sitravatinib, R428, TP0903, BMS-777607, NPS1034, MRX-2843, UNC2025, UNC3133, ONO-7475, Tyro3 inhibitors, 5F9, and combinations thereof.

18. The method according to claim 16, wherein the liquid biological sample is selected from the group consisting of blood, serum, plasma, urine, breastmilk, saliva, tears, and sweat.

19. The method according to claim 16, wherein the cancer characterized by high surface PS expression is selected from the group consisting of pancreatic cancer, glioma, melanoma, lung cancer, colorectal cancer, and pediatric cancers.

20. The method according to claim 16, further comprising administering to the subject one or more additional anti-cancer therapeutics selected from the group consisting of an Hsp70 inhibitor, a Toll-like receptor 2 (TLR2) inhibitor, and a Mer tyrosine kinase (MerTK) inhibitor, in combination with the one or more anti-cancer therapies targeted to high surface PS expressing-cancers.

21. The method according to claim 1, wherein high surface PS-expressing cancer cells are cells having an Annexin V fluorescence of greater than or equal to about 2000 MFI; and wherein low surface PS-expressing cancer cells are cells having an Annexin V fluorescence of less than or equal to about 1500 MFI.

22. The method according to claim 16, wherein high surface PS-expressing cancer cells are cells having an Annexin V fluorescence of greater than or equal to about 2000 MFI; and wherein low surface PS-expressing cancer cells are cells having an Annexin V fluorescence of less than or equal to about 1500 MFI.
